(19) United States
(12) Patent Application Publication
(10) Pub. No.: US 2024/0420156 A1
(43) Pub. Date: Dec. 19, 2024

(54) SMART LABELING MANAGEMENT

(71) Applicant: IQVIA Inc., Parsippany, NJ (US)

(72) Inventors: Cham WILLIAMS, Media, PA (US); Deven MEHTA, Belle Mead, NJ (US); Julian BACKHOUSE, Wirral (GB); Gilberte HOUBART, Cambridge, MA (US); Guilherme COUTO, Rio de Janeiro (BR)

(21) Appl. No.: 18/333,973

(22) Filed: Jun. 13, 2023

(51) Int. Cl. G06Q 30/018; G06Q 10/0633; G16H 40/20

(52) U.S. Cl. CPC G06Q 30/018; G06Q 10/0633; G16H 40/20

(57) ABSTRACT

Methods, systems, and apparatus, including computer programs encoded on computer storage media, for managing a life cycle of a label. In some implementations, a request for generating a label for a product can be received. Workflows for generation of the label can be identified. The workflows for the generation of the label can be executed. In response to executing the workflows for the generation of the label, data indicative of the generation of the label can be submitted to a health authority. Data indicative of the approval of the label for the product can be received from the health authority. In response to receiving data from the health authority indicative of approval of the label for the product, a layout for the label can be generated for the product.

### TECHNICAL FIELD

This specification generally relates to digital label management using interface systems.

### BACKGROUND

Labels provide users of a product with information about the product. A digital version of the label can be, for example, a machine-readable label or a human readable label that contains information about the item to which it is attached. Practically, the label can point to an external location that can store or reference information about the product to which it is attached.

### SUMMARY

The subject matter of this application describes a system that can manage and visually display real-time information regarding end-to-end processes of a life cycle for a label. The system can include one or more servers that communicate with various end devices requesting label creations. The system can receive a request for creation of a label for a product from each of the various end devices, execute the processes for creating each label based on the respective request, and provide status information for each label creation request through a visual display. Moreover, the system can submit an approved label creation to a health authority for regulatory approval. The health authority can approve the label creation based on rules and regulations pertaining to a location of the health authority. In response to receiving the regulatory approval from the health authority, the system can perform the processes for generating the approved label and translating the generated label into other languages depending on one or more locations where the product associated with the label is to be sold. The system can focus on monitoring a heavily regulated process of label creation for products in various industries. These various industries can include, for example, the pharmaceutical industry, the medical device industry, the food industry, and other industries, to name some examples. In some examples, the system can be adaptable and flexible in showing support to performing the label creation process in various countries and regions, each with its own process and rules. For instance, in Europe, there is a specific “clock,” e.g., 30 days, 60 days, to get through specific phases in the label creation process. The system can incorporate the timing for a specific geographic region as defined by a local Health Authority.

In some implementations, the system can perform various functions related to the end-to-end processes of a label's creation. In further detail, the system can incorporate various modules or products that form together. In some examples, the system can utilize content management for the actual documents and the workflows to manage them as part of the label creation process. In some examples, the system can utilize labeling to manage the label requesting process across the platform from end-to-end. In some examples, the system can utilize a platform to manage the various databases, e.g., drug databases, medical databases, and pharmaceutical databases, the core data of the label creation process, and the administration functions, e.g., application configuration, user permissions, and other. The system can combine the content management module and the platform module to manage regulatory and other domain documents as a point solution. In some examples, the system can combine the content management module and the platform module to be integrated with other vendors or other publishing tools.

These functions can include processes related to label requests, document workflows associated with the label request, label content localization, submission to a health authority, artwork management, and dashboard management. As will be further described in detail below, each of these processes can be monitored by users through a dashboard in real-time. Users can also submit requests from their end devices to the system to view status information regarding the label's life cycle. In some implementations, the system can process multiple label requests at a time. In doing so, the system enables multiple users to visually inspect and monitor the status of their different label creations in real time.

The function for processing label requests can include receiving label requests from one or more end devices indicative of a desire to have a label created. The label requests can include requests for labels at the core, regional, or local levels, each of which will be further described below. In some implementations, the system can analyze the request to determine whether the request includes the correct information pertaining to the product being created during an impact assessment phase. Users, machine processes, and other various processes, to name a few examples, can analyze characteristics related to the request and determine whether the request is approved or denied. In response to analyzing the request, the system can either approve or deny the label request. In some examples, a user may assist the system during the approval or denial of the label request. If the label request is approved, the system can initiate the processes to perform document workflows. If the label request is denied, the system can notify the user and cease processing or can resubmit the label request with additional information added to the revised label request.

In some implementations, the system can perform the processes related to document workflows. A workflow can represent processes related to managing document creation and document updates. In some examples, the system can enable different types of workflows. For example, the different types of workflows can include authoring a document, reviewing a document, approving of a document, and translating of a document. A workflow can be setup or instantiated by a managing user, such as a document coordinator. The workflow is typically assigned to one or more assignees by a document coordinator or another user with the set level of permission. Some workflows can be initiated as automatically as part of a “multiworkflow” process. For example, by combining authoring, review, and approval workflows.

Specifically, the creation of a label includes managing multiple templates used to create documents and requests, among others, verifying data on the documents, and the duration of processes related to those documents. For example, a drug label or a medical device label can include various pieces of information such as drug dosage, drug composition, name, country, or region where it is distributed, and other information. The system can auto populate multiple templates with the information from the request and execute these templates through various workflows to perform the label creation.

A document coordinator manages the various workflows in a document management system. Specifically, a document coordinator can setup workflows, track workflow status of each task assignee in the workflow of the document management system. Additionally, the document coordinator can designate durations for the completion of each task. The document management system enables the document coordinator in visualizing an amount complete for a workflow of the task assignee. As will be further described below, a workflow can be completed when each of the assignees, including the document coordinator, have marked or signaled their tasks as completed. The document management system can receive a chain of notifications customized to user profiles from each of the task assignees to aid the document coordinator in determining when a workflow is complete.

The different tasks designated by the document coordinator for a workflow can include, for example, authoring the document or packets of documents, reviewing the documents by experts to assert validity and compliance, and approving the final documents version before it moves on to the next step. Other tasks are also possible and will be further described below.

The document coordinator monitors various workflows, e.g., including “multi-workflows” which are single workflows linked in a sequence, typically author-review-approve). In further detail, the document coordinator, or a business administrator, can define workflow templates prior to receiving a label request or prior to being assigned to work on non-Label Documents. Templated workflows help speed up the process for Document Coordinators to set up workflow. The type of tasks assigned in Content Management can include, for example, edit, review, approve. The Label Request process can rely on a Planner, e.g., a Gantt project management tool, to track Stages and Milestones. By relying on templated workflows and tracking the completion of workflows, the system can provide increased productivity to clients as well as to the label creation process itself. Tracking cycles enable teams, e.g., global teams or local leads specifically, to measure a frequency amount of re-entering a corresponding step, e.g., as a label request moves through the steps, due to issues with the step. Consequently, the system can seek to minimize re-cycling or minimizing the frequency with which the same step for the label request process is performed.

In some implementations, the system can perform label content localization. Label content localization can include translating generated data associated with a label to a specific language. The request provided by the end-user, e.g., such as a Global Label Lead (GLL), can designate one or more locations where the product associated with the created label is to be sold and/or distributed. The system can determine whether the language of the one or more designated locations requires language translations. As such, the system can perform translations of generated labels for the product to any language where the product is to be sold.

In some implementations, the system can generate and securely store artwork to be utilized with the label. The system can include one or more automation engines that can auto generate artworks to be included with the label. For example, the artworks can include pharmaceutical artworks, medical artworks, food-packaging artworks, and other types of artworks. The artworks can include words that need to be translated during the label content localization based on locations where the product is to be sold, e.g., translating the label content from “English” to “French”. Once the artwork has been generated and translated, the system can store the generated and translated artwork in a blockchain for artwork security and validation.

In some implementations, the system can display a user interface that enables a user to monitor the end-to-end process of the label creation life cycle. In some implementations, the system can display the user interface that enables the user to monitor the end-to-end process of a label update. Specifically, the user interface includes an activity dashboard that includes one or more user interfaces for displaying information relating to label management, authoring, content management with review, approval and reporting, and label reporting into a single system. Moreover, the activity dashboard can display items relevant to a user based on their respective profile, e.g., one or more label requests, documents, workflows, tasks, notifications, and related metrics. The activity dashboard can include one or more interactive elements that enable a user to view statuses of the label creation or updating. In some examples, the activity dashboard can provide an illustrative storyboard view of the workflow processes against one or more time constraints. For example, the activity dashboard, which tracks multiple requests and their associated workflows and documents, can display a Gantt chart for a specific request. In this example, the Gantt chart can illustrate a project schedule with one or more graphical interfaces, e.g., a bar chart, a list view, or other, showing the tasks to be performed on the vertical axis, and time intervals on the horizontal axis. The width of the horizontal bars of the Gantt chart can represent a duration of each activity for a particular task. The Gantt chart can display milestones and tasks completions related to the label creation/update life cycle.

Some functions of the system include, for example, viewing full lists of documents and requesting different list views. The system can present the full lists of documents according to permissions associated with a user profile; creating saved searches to review all active filtered items; and, coordinating via built-in tools with teams to review for instance specific changes to a document. For example, documents can be annotated and assigned users can create and review annotations. In some examples, task assignees can interact with the system via emails, networking applications, text messaging, and other forms of communication. By providing a system that enables a document coordinator or other to view status of workflows, the system can track the statuses and provide updates regarding a label creation request within one location when the tasks of the workflows may be performed in disparate locations.

In one general aspect, a method performed by one or more computing devices includes: receiving, by one or more processors and from a first client device associated with a user, a request for generating a label for a product; identifying, by the one or more processors, one or more workflows for the generation of the label; executing, by the one or more processors, the one or more workflows for the generation of the label; in response to executing the one or more workflows for the generation of the label, submitting, by the one or more processors, data indicative of the generation of the label to a health authority; receiving, by the one or more processors, data indicative of approval from the health authority of the label for the product; and in response to receiving data from the health authority indicative of approval of the label for the product, generating, by the one or more processors, a layout for the label of the product.

Other embodiments of this and other aspects of the disclosure include corresponding systems, apparatus, and computer programs, configured to perform the actions of the methods, encoded on computer storage devices. A system of one or more computers can be so configured by virtue of software, firmware, hardware, or a combination of them installed on the system that in operation cause the system to perform the actions. One or more computer programs can be so configured by virtue having instructions that, when executed by data processing apparatus, cause the apparatus to perform the actions.

The foregoing and other embodiments can each optionally include one or more of the following features, alone or in combination. For example, one embodiment includes all the following features in combination.

In some implementations, receiving the request for generating the label for the product further includes: extracting, by the one or more processors, data from the request representing the label for the product; submitting, by the one or more processors, the extracted data from the request to impact assessors to analyze socioeconomic significance of distribution of the product associated with the label; providing, by the one or more processors, data indicative of a first status to a dashboard display indicative that the request has been submitted to the impact assessors; receiving, by the one or more processors, data indicating of approval from the impact assessors for distribution of the product associated with the label; and providing, by the one or more processors, data indicative of a second status to the dashboard display indicative that the request has been approved by the impact assessors.

In some implementations, identifying the one or more workflows for the generation of the label further includes: assigning, by the one or more processors, a document coordinator to the one or more workflows for processing the generation of the label; receiving, by the one or more processors and from the document coordinator, data indicative of one or more tasks for the one or more workflows, data indicative of one or more assignees for performing each of the one or more tasks, and data indicative of a length of time each of the one or more assignees has been designated for performing each of the one or more tasks; determining, by the one or more processors, a complexity likelihood for each of the one or more workflows using a trained machine-learning model; comparing, by the one or more processors, the complexity likelihood for each of the one or more workflows to a threshold value; determining, by the one or more processors, whether the complexity likelihood for each of the one or more workflows satisfies the threshold value; and in response to determining the complexity likelihood for each of the one or more workflows satisfies the threshold value, designating, by the one or more processors, the one or more workflows to be performed in a multi-workflow process.

In some implementations, wherein the trained machine-learning model is configured to (i) receive as input: the data indicative of the one or more tasks for the one or more workflows, the data indicative of the one or more assignees, and the data indicative of the length of time each of the one or more assignees has been designated for performing each of the one or more tasks; and, configured to (ii) output: the complexity likelihood indicating whether a complexity of the one or more workflows is high complexity or low complexity.

In some implementations, determining whether the complexity likelihood for each of the one or more workflows satisfies the threshold value further includes: determining, by the one or more processors, the one or more workflows to be of high complexity in response to determining the complexity likelihood for each of the one or more workflows satisfies the threshold value; or determining, by the one or more processors, the one or more workflows to be of low complexity in response to determining the complexity likelihood for each of the one or more workflows does not satisfy the threshold value.

In some implementations, the method further includes in response to determining the complexity likelihood for each of the one or more workflows does not satisfy the threshold value, designating, by the one or more processors, the one or more workflows to be performed in a single-workflow process.

In some implementations, wherein executing the one or more workflows for the generation of the label further includes executing the one or more workflows for the generation of the label using either the single-workflow process or the multi-workflow process.

In some implementations, the method further includes: in response to executing the one or more workflows for the generation of the label further comprises: providing, by the one or more processors and to a dashboard display, data indicative of status for each of (i) the one or more workflows, (ii) the layout for the label of the product, (iii) an expected time duration for completing each of the one or more workflows, and (iv) a risk associated with completing each of one or more of the workflows; and receiving, by the one or more processors, user interactions with data displayed on the dashboard display from a second client device associated with the user.

In some implementations, wherein providing data indicative of status for each of (i) the one or more workflows, (ii) the layout for the label of the product, (iii) an expected time duration for completing each of the one or more workflows, and (iv) a risk associated with completing each of the one or more workflows, to the dashboard display further includes: displaying, by the dashboard display, the status for each of (i) the one or more workflows, (ii) the layout for the label of the product, (iii) an expected time duration for completing each of the one or more workflows, and (iv) a risk associated with completing each of one or more of the workflows; and displaying, by the dashboard display, real time updates related to the execution of the one or more workflows.

In some implementations, wherein submitting the data indicative of the generation of the label to the health authority further includes: receiving, by the one or more processors, an indication that the one or more workflows have successfully completed; designating, by the one or more processors, a health authority to submit the data indicative of the request using extracted data from the request; generating, by the one or more processors, a data package indicative of (i) results from performing the one or more tasks for the one or more workflows, (ii) data extracted from the request for the label, and (iii) data indicative of approval from impact assessors for distribution of the product associated with the label; submitting, by the one or more processors, the data package to the designated health authority; and providing, by the one or more processors, data indicative of a third status to a dashboard display indicative that the request has been submitted to the designated health authority.

In some implementations, wherein receiving the data from the health authority indicative of approval of the label for the product further includes: providing, by the one or more processors, data indicative of a fourth status to a dashboard display indicative that the request has been approved by the health authority; storing, by the one or more processors, data indicative of artwork from the request in a blockchain network for validation; and generating, by the one or more processors, data indicative of the label from the request for distribution, wherein the data indicative of the label comprises (i) the artwork for the label from the request, (ii) the data indicative of the label, and (iii) a layout of the label.

In some implementations, the method further includes: transmitting, by the one or more processors, the data indicative of the label from the request to a manufacturer; receiving, by the one or more processors, data indicative of the artwork generated by the manufacturer; comparing, by the one or more processors, the data indicative of the artwork generated by the manufacturer to artwork stored in the blockchain network to validate that the artwork generated by the manufacturer is being properly generated; and in response to determining that the artwork generated by the manufacturer successfully validates to the artwork in the blockchain network, transmitting, by the one or more processors, a notification to the manufacturer indicating that the artwork is successfully validated.

In some implementations, the method further includes: in response to determining that the artwork generated by the manufacturer does not successfully validate to the artwork in the blockchain network, transmitting, by the one or more processors, edits to the artwork being generated by the manufacturer.

The subject matter described in this specification can be implemented in various embodiments and may result in one or more of the following advantages. In some implementations, the system can consolidate label information for various label requests. The system can consolidate label information for various label requests can be monitored and tracked in a single platform. In this manner, the platform can provide transparency for the various workflows for the label generation and track the various core to local requests and artwork implementation. The system can also provide increased collaboration and orchestration from workgroups to national and international labeling teams.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A is a block diagram that illustrates an example of a smart label management system.

FIG. 1B is a block diagram that illustrates an example of modules for processing a label request of a smart label management system.

FIGS. 1C-1E are flow diagrams that illustrate examples of a workflow process of a smart label management system.

FIG. 1F is a block diagram that illustrates an example of modules for health authority submission of a smart label management system.

FIG. 1G is a block diagram that illustrates an interface for monitoring a status of a label request in a smart label management system.

FIGS. 2A-2J are block diagrams that illustrate interfaces for interacting with a smart label management system.

FIG. 3 is a flow chart that illustrates an example process for executing a label request with a smart label management system.

FIG. 4 is a block diagram of a computing system that can be used in connection with methods described in this document.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

Current labels for products are subject to a number of issues. In some examples, creating and/or updating current labels for products can be a lengthy and complex process and error prone. Adding more complication, when a label is generated for a specific country, that country imposes their own specific rules and regulations. As a result, each country imposes regulations on this process, requiring the system described throughout this specification to track and accommodate each of the rules and regulations for each of the countries.

In some examples, another challenge relates to the strict structure of regulated documents associated with the generation of a label. Each section of a document is required to obey certain rules and guidelines. In particular, individual sections of a document have become—as technology evolves—a “component” that requires versioning and can be reused. Consequentially, an author can be required to have access to each of these components. The system described in this specification can streamline the entire process of receiving a label request to generating a label, tracking the documents accompanying the generation of the label, tracking the components in the documents and their corresponding versions, and gathering and tracking each of the documents renditions, e.g., in various formats, as well as the relationships between documents that need to be grouped.

First, the generation of a label can require identification and location of a large amount of documents to be either updated or attached for reference. Documents that are required to be updated or attached for reference can include, for example, results of clinical trials to support the safety of a new drug. In this example, experts are required to review the results of the clinical trial to ensure the appropriate information is included. Second, the generation of the label can require ensuring that updates to the documents that are being revised are accurate, consistent with regulations, and properly sequenced to support submission regulations. The system can engage key personas, such as the Global or Local Leads, on a daily basis, or other basis, in coordinating all aspects of the process. The generation of the label can produce a large number of forms that are required to be monitored and tracked. For example, the large number of forms can be utilized in updating a Drug Database or in classifying appropriately any document in the documents database. Each document can include various fields that need to be tracked according to version control. These fields can represent the “metadata” associated with a document.

In some implementations, the generation of the label requires a publishing process to a local Health Authority. The system can gather each of the documents created and monitored for the generation of the label and can publish each of these documents to the local Health Authority. The process of gathering and publishing each of these documents is complex in that a multitude of tree structures of folders that are themselves regulated to host the documents are required to be provided to the local Health Authority. The local Health Authority reviews each of the documents and the tree structure of folders to review the submission of the label creation. The system described in this specification provides an easy-to-use interface to optimize the process of adding documents to a large number of folders, and subsequently publishing this information to the local Health Authority. In further detail, the system can include a Publishing Manager or a Submission Manager that is responsible for ensuring the publication of documents to the local Health Authority is performed in a timely manner and according to specific rules and regulations.

In some implementations, smart label management can refer to platforms that collect drug label information and other data in support of the drug label information. The information can include, for example, blockchain information, health regulatory information, drug label creation, drug label digitization, workflow processes, and drug label updates for easy authorization and processing of necessary updates to drug label concerns. However, digital drug labels may not be typically tracked from their originating source, e.g., clinical trials through regulatory approval, and through to manufacture, supply, and distribution of the drug. Typically, digital labels may also not be tracked through to any updated data sources, through blockchains, or through other mechanisms based on authorized individuals responsible for the digital label.

The techniques described in this specification enable the monitoring, tracking, and interacting with a lifecycle of a label request associated with a product. In further details, the techniques include monitoring, tracking, and interacting from the creation of a label request, e.g., a trigger event such as a safety warning, to the creation of the label itself, to regulatory approval at local and global levels, and prior to the distribution of the label for product's release. In some implementations, the techniques can be related to products separate from or in addition to prescription drugs. For example, the request can be associated with a food product, a medical device, and devices of other industries, to name examples.

In some implementations, the system described enables integration, creation, and monitoring of the end-to-end process of a drug label request. The processes can include, for example, a label request, a document lifecycle, label content localization, submission to a health authority, and creation and maintenance of artwork management. More specifically, the system integrates the tracking, creation, and monitoring of the label management on a single platform that can be monitored by one or more external users. The system can receive one or more label requests from various end user devices and perform the following: (i) assess the label requests to determine whether they are approved or denied for processing, e.g., an Impact Assessor can be responsible for making the determination; (ii) assign to the Document Coordinator the labels to be created or updated e.g., this can be handled by the Content Management module, and aid executing this process with tracking and activating user actions in context; (iii) submit the approved label updates or new labels for new products, e.g., drugs, to the various health authorities where the product, e.g., drug or medical device, is distributed); (iv) distribute the approved labels and artwork for packaging approved by various the health authorities; and, (v) provide monitoring and status updates regarding the label creation in real-time throughout the end-to-end process. The monitoring and status updates regarding the label creation in real-time can include, for example, prompting the user with the appropriate action in a given context and status. The system can rely on various machine-learning processes to improve the processing of the label requests, as will be further described below.

For this system, the product, e.g., drug, medical device, or other comes with physical packaging and inserts (aka “artwork) displaying content that needs to be compliant with local regulation for the market where they are distributed. The text language contained within regulated documents are either managed internally as the company's position on any drug or device (never within the public domain) or as local and regional labels (which are submitted to the health authority and eventually approved. That approved text is what appears within a patient or prescriber leaflet, medical guide and upon self-adhesive pill tub labels, cartons and other packaging items.

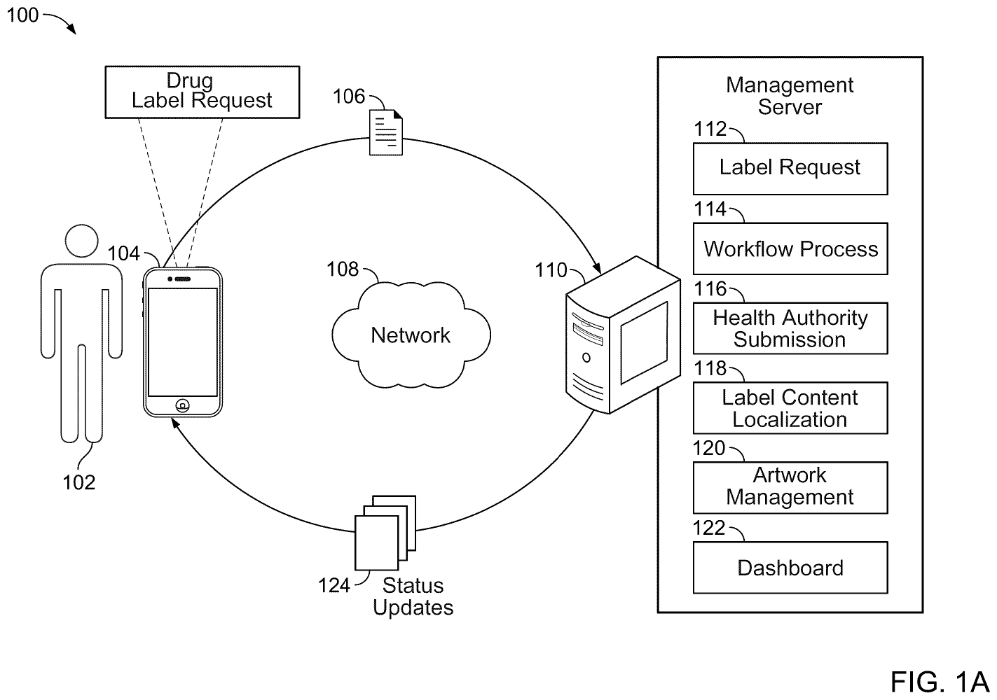

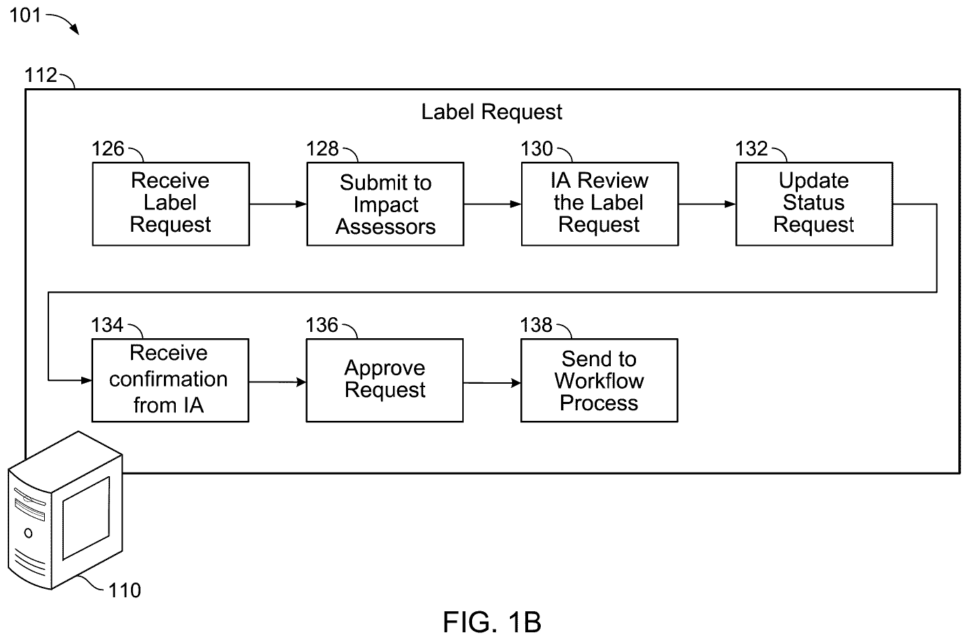

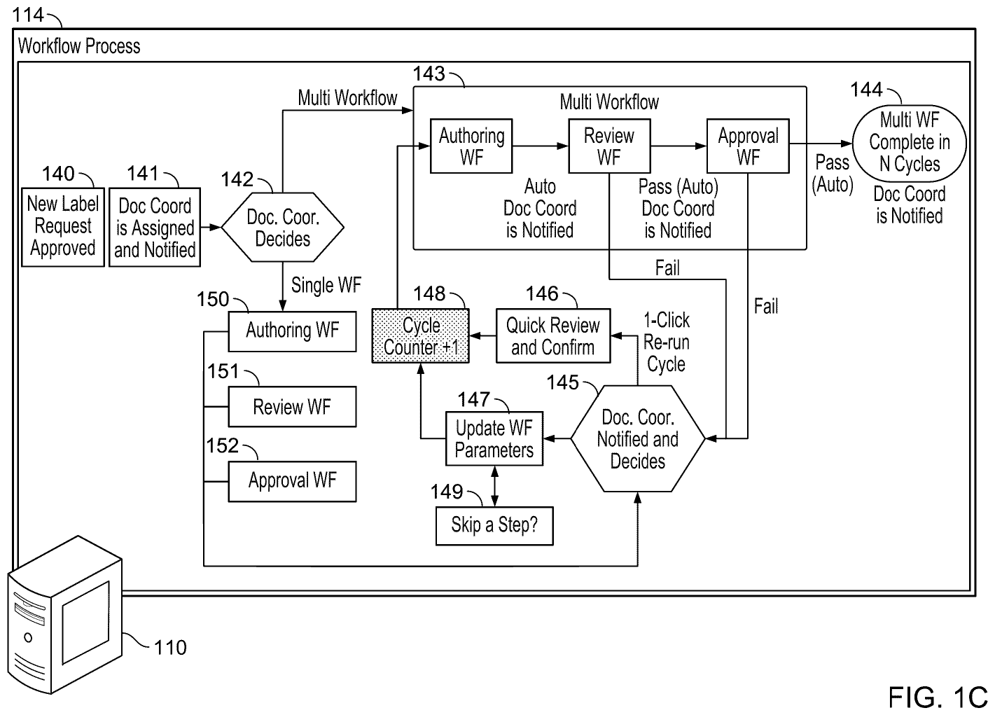

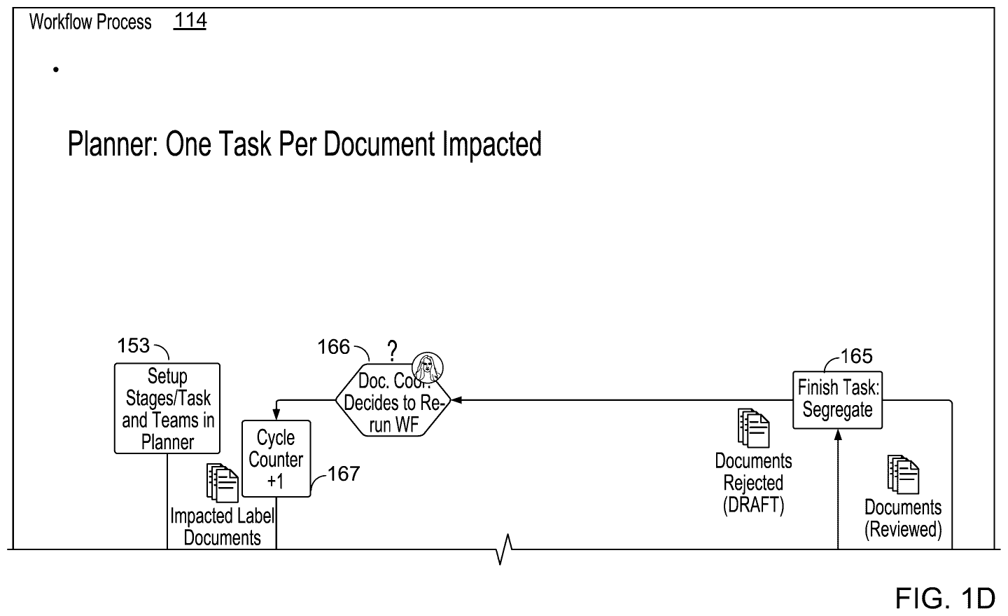

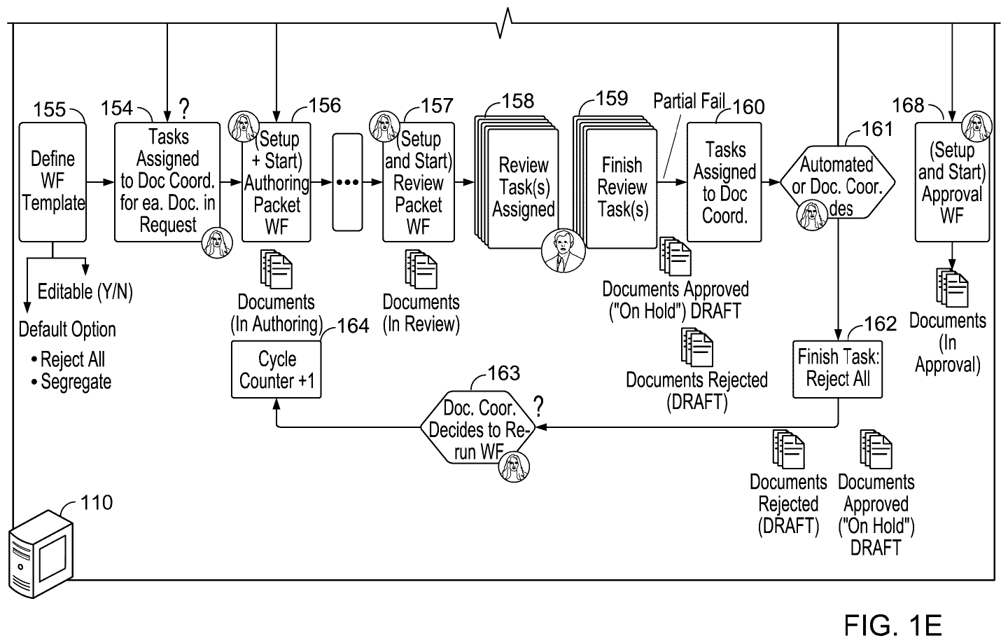

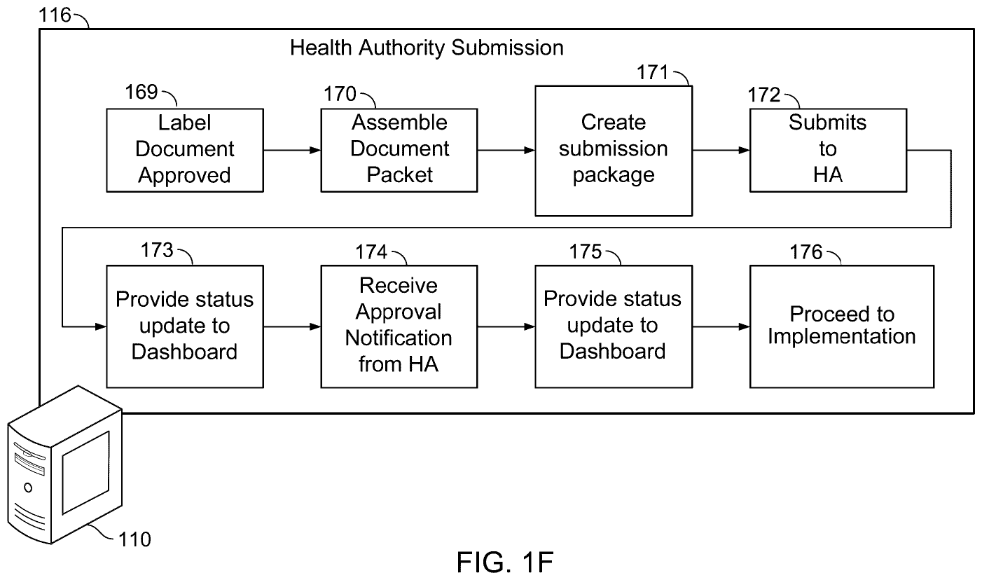

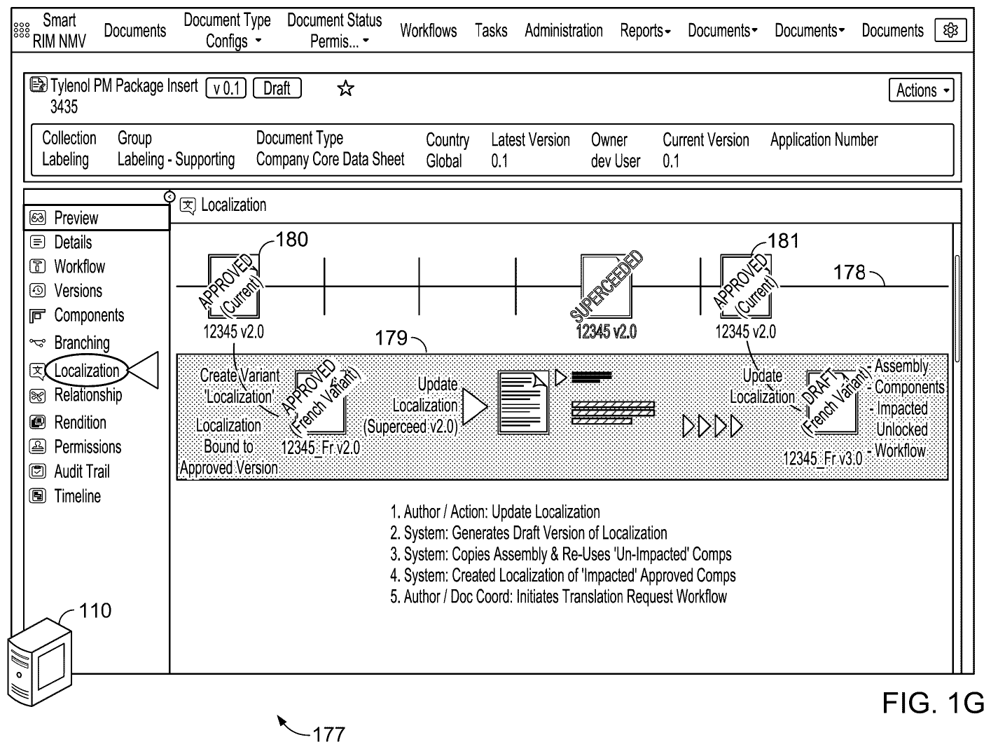

FIG. 1A is a block diagram that illustrates an example of a smart label management system 100. The smart label management system 100 can be a predictive system configured to process a label request and determine whether the label request is approved for creation. Specifically, the smart label management system 100 can be trained to identify contents of the label request provided by a user and determine whether the label request is to be approved by a health authority. Additionally, the smart label management system 100 can provide status updates to the submitting user during the creation of the label, communicate with various health authorities regarding approved labels, assign tasks for various workflows associated with the label generation, and generate data indicative of the label request to provide to the submitting user.

In general, the smart label management system 100 can support the creation of a label request from a submitting user, e.g., a Global Label Lead (GLL), a Local Label Lead (LLL), or another individual, and generate data indicative of statuses of workflows or documents associated with the label creation request. The smart label management system 100 can support a database query to find documents, e.g., labels or other, requests, workflows, and drug products. The smart label management system 100 can operate within a safety or change event tracking system either connected or not connected to the smart label management system 100.

In some implementations, the smart label management system 100 can communicate with external health authorities to seek approval of the submitted label by providing each of the accompanying documents. While performing the creation of the label, the smart label management system 100 can provide updates to a dashboard that display data indicative of the label request, document and workflow statuses, and actions available to the user in supporting contexts, which can dynamically change. FIG. 1A illustrates various operations that can be performed in various stages, which may be performed in the sequence indicated or another sequence.

In some implementations, the smart label management system 100 can include a client device 104, a management server 110, and various databases. The management server 110 can include one or more computers connected locally or connected over a network. The one or more computers of the management server 110 can include different application programmable interfaces (APIs), one or more processors, and different modules for performing functions related to the creation of the label request. The management server 110 can be configured to communicate with client device 104 over network 108. The network 108 can include for example, a local network, such as Bluetooth or Wi-Fi, a larger network, such as an Internet, or a combination of a local and a larger network. Alternatively, a user, e.g., user 102, may directly interact with the management server 110 by way of a touchscreen, a monitor, keyboard, and mouse, or actively speaking with the management server 110.

The management server 110 may include one or more computers connected locally or over a network. The management server 110 may communicate with various databases, various health authority systems, various distributed systems, and other client devices. The management server 110 can communicate with the client device 104 for various functions. These functions can include, for example, obtaining a query regarding a label creation request, requesting for additional details from the client device 104 regarding the label creation, indicating whether the label creation request is approved or denied to the client device 104, notifying the client device of the submission of the approved label creation to one or more health authorities, notifying the client device of processing the approved label creation by assigning workflows to various assignees, and providing results of the label creation to the client device corresponding to the submitting user, to name a few examples. Moreover, the management server 110 can provide a dashboard that enables the submitting user 102 to view status updates of the label creation process in real time through an application on their client device or a connected display to the management server 110 in real time. The management server 110 can also perform other processes, as will be outlined and described below.

In some implementations, a user 102 can submit a request for a label creation to management server 110. Specifically, the user 102 can interact with a client device 104 and provide data related to the label creation. In some examples, the user 102 can provide data related to the label creation using an email, text messaging, file transfer protocol, audio messages, and physical mail to the management server 110.

In some implementations, data related to the label creation can include data pertinent to a type of label being created. For example, if the label is related to a drug, such as a new drug or updates to an existing drug, then the data related to the label creation can include data related to the early phases of the drug's lifecycle. This may include data related to the development and testing of the drug, such as drug composition, symptoms, side effects, compound plans, clinical trial results, efficacy study results, safety warnings, expected therapeutic area (TA), target plan, safety test results, government entries, data relevant to electronic common technical document (eCTD) regulatory filing, data regarding which personnel were involved in the design manufacture, approval, and sale of the drug. Additionally, the data related to eCTD filing can include contents of the filings themselves, as well as details that surrounding the approval of the drug, reasons for regulatory approval, tests run, necessary/recommended warnings for the drug, and data indicative of regulatory personnel involved in any of the aforementioned activities.

If the label or document to be created or updated relates to a medical product, then the data related to the documents and label request can include product development data, release dates of the product, compounds used within the product, processes used to create the product, safety test results, sale of the medical product, and personnel designed to use the medical product, to name some examples. Other information can include markets, e.g., country or region, for distributing the medical product. Other types of products and devices can also utilize the smart label management system 100.

In some implementations, the label request 106 can include additional information to be transmitted to the management server 110. The additional information can include information about the user 102, such as credentials of the user 102 for authorization to communicate with management server 110. The credentials of the user 102 can include a username and a password, data identifying the client device 104, data identifying a user profile of user 102, and location of the client device 104, to name some examples.

The additional information can also include data indicating whether the label request is to be processed on an expedited or standard process. The expedited process can indicate to the management server 110 that the label creation request is to be performed on an accelerated track, e.g., less than 3 months, for example. In some examples, the expedited process may require the user 102 to provide financial payment for approval of the accelerated track. The standard process can indicate to the management server 110 that the label creation request is to be performed on a normal timeline without additional financial payment.

The client device 104, which can include a personal handheld device, a mobile device, a tablet, a personal computer, or other, receives the user 102's input, packages the inputs, and transmits the label request 106 to the management server 110 over the network 108. Alternatively, if the client device 104 is connected to the management server 110, e.g., a display for user interaction connected to the management server 110, and then the management server 110 can accept the label request 106 in response to the user 102's entry in client device 104.

In some implementations, the management server 110 can receive the label request 106 from the client device 104. In some implementations, the management server 110 can recognize and determine that the label request 106 corresponds to a request from particular user, e.g., user 102. For example, the management server 110 can first authorize the user 102's access by comparing stored credentials to credentials provided in the label request 106. If the management server 110 determines that a match does not exist, the management server 110 can deny user 102's access to submit the label request 106. Alternatively, if the management server 110 determines that a match does exist, the management server 110 can then match the user 102 to a particular stored profile using the credentials provided by the user in the label request 106 or the matched stored credentials in the management server 110's memory.

In some implementations, the management server 110 can include various modules that can perform the processes related to the label request. Specifically, the modules can include label request 112, workflow process 114, health authority submission 116, label content localization 118, artwork management 120, and a dashboard 122. The management server 110 can also include other modules such as, for example, user profile management, application configuration settings and blockchain management. Each of these modules can perform various functions related to the process of the label update or creation. These modules can be performed by one or more automated APIs, machine-learning programs, and automated tasks. Additionally, various parties, various individuals, and various departments can perform the tasks associated with the label request.

The label request 112 can include receiving label requests submissions from one or more client devices that desire to have a label created. The label requests can include labels at the core, regional, or local levels. In some examples, the system can support the assessment by an Impact Assessor of the label request 106 to determine whether the request includes the correct information pertaining to the product being created or updated during an impact assessment phase. In some examples, users, machine processes, and other various modules, to name a few examples, can analyze characteristics related to the request and determine whether the request is approved or denied. In response to analyzing the request, the system, e.g., the Impact Assessor or one or more machine processes, can either approve or deny the label request. If the label request is approved, the system can initiate the processes to perform document workflows by notifying the Document Coordinator, assigning them the task and related documents. If the label request is denied, the system can notify the user 102 that created the label request with the reasons for rejection at which point the cycle could be restarted.

As the label creation request moves through the processes of the smart label management system 100, the smart label management system includes various stages that correspond to the lifecycle processes. The stages include a request lifecycle, a core request lifecycle, a local-national request lifecycle, a local-regional parent request lifecycle, and a local-regional child request lifecycle. The request lifecycle can include a planner, in which core, local, and regional request lifecycles move through stages of a request plan, which can be configured within the system administration area.

The core request lifecycle can include a global impact assessment, a label request creation, label request assessment, label request confirmation, core label workflow, e.g., managing the lifecycle of the document, core label distribution, and acknowledgment. The local-national request lifecycle can include local impact assessment, label request creation, label request assessment, label request confirmation, local label workflow, submission preparation, health authority submission, final health authority submission, health authority decision, and receiving health authority decision. The local-regional parent request can include the local impact assessment, label request creation, label request assessment, label acknowledgement, and label request confirmation. The local-regional child request can include local impact assessment, label request creation, local label workflow, submission preparation, health authority submission, final health authority submission, health authority decision, and receive health authority decision.

In some examples, the system when in a stand-alone configuration allows for users either at the global ‘core’ label level or at region, market, local country level, to initiate a change to one or multiple labels. For example, if a change to a label is required at a local level in the United Kingdom (UK), the change can be raised, assessed, and the changes processed. Then, the system can submit the changes to the local authority, e.g., the Medicines & Healthcare Products Regulatory Agency (MHRA). Once the MHRA local authority approves the label changes, the system can update artworks associated with the label change and the system can create new packaging commodities. Notifications to the global lead can take place, informing the global lead that changes have taken place to the UK local label for the product or drug associated with the label. This change acts as the trigger for the global lead to review the changes made by the UK and to update the corresponding core labeling. The smart label management system 100 can propagate these changes to countries other than the UK, unless additional changes have taken place at the core, during which time these other countries would receive the trigger or notification and perform the corresponding impact assessment for their label.

In some examples, core requests can manage the creation or changes to an existing core label document or set of documents. These set of documents are internal and are not distributed outside of the smart label management system 100. In an example, a label request in the EU can be performed for a drug. A lead country from within the EU block, e.g., Germany, for example, can process changes on behalf of the other member states. The member states are in observation mode while the lead country gains approval with the health authority in the EU, e.g., European Medicines Agency (EMA). Once approved by the EMA, each member state can localize the approved ‘Regional’ label and negotiate that translation with their countries health authority, e.g., for France, converting a label in German to French. The smart label management system 100 in this example can manage local level requests, which represent requests that are specific to a particular region or country.

In some implementations, changes to a core label for a drug can affect label changes in other countries. For example, if changes to a core label for a drug have been approved in the EU, then labeling, artwork, and packaging for other countries are impacted by the core's changes. Australia, for example, would require labeling, artwork, and packaging changes for the drug as impacted by the core changes. In this example, Australia's local lead can receive a notification of the core label change for the change and Australia's local lead can acknowledge that they have read the updates to the core. Next, Australia's local lead can generate a local label request, identify impacted labels by the core label change, and the local label lead can submit the request for approval in Australia. In some cases, a core change for a label can spark events that see regional changes taking place in the EU for 26/7 member states, and for single examples such as with Australia, Nigeria, Japan, and so on as related single label requests.

Additionally, the label request 112 can provide status updates to the user 102 that submitted the label request 106. As will be further described below, the user 102 can interact with the management server 110 through dashboard 122. The dashboard 122 can include one or more graphical user interfaces (GUIs) that enable user 102 or other user to monitor and/or track a status of a label creation request pertaining to the label request 106. As such, the user 102 can view the dashboard 122 of the management server 110 to monitor a status of the label creation request.

The workflow process 114 can include functions related to setting up and executing various workflows related to the approval of the label request 106. A workflow can correspond to processes related to managing, authoring, reviewing, and approving different documents related to the label request. For example, the management server 110 can perform three different types of workflows-authoring a document, reviewing a document, and approving of a document. In some implementations, a workflow related to a label request can be setup and instantiated by a managing user, such as a document coordinator. The managing user can assign tasks associated with the workflow to one or more assignees to perform the tasks.

Additionally, the document coordinator can assign one or more assignees to perform multiple tasks of a workflow. For example, the document coordinator, which can be a person, can instruct an assignee to create one or more templates using information from the label request 106, review data filled in the templates using information from the label request 106, and approve data filled in the templates using information from the label request 106. Other tasks of a workflow can include assigning tasks to assignees, authoring a workflow packet of various templates, reviewing workflow packets, reviewing assigned tasks, rejecting document workflows, and approving workflows. A workflow packet is an object that contains one or more documents moving through a workflow together. The management server 110 supports situations where a workflow packet can be segregated at the end of an approval workflow either automatically via workflow settings or by the Document Coordinator. The Document Coordinator can decide to manually reject the whole workflow packet, allow an approved portion of the workflow packet to continue its cycle, and the rejected portion of the workflow packet to repeat another round of review and approval. For example, a workflow packet can include five documents, and each document would need to reach an approved state for the packet itself to be approved. In this example, an author may work on, e.g., review, a single document of a packet attached to the workflow. The management server 110 can monitor the workflow tasks with a cycle counter, determine whether to execute a multi-workflow process, and determine whether to execute a single workflow process.

Ultimately, the management server 110 can submit these templates or forms to one or more health authorities as designated by the label request 106. As the assignees perform and/or complete the tasks assigned by the document coordinator, the assignees can provide status updates of their tasks to the management server 110. In this manner, user 102 can view through the dashboard 122 a status of their label creation request in real time as the label is reviewed and created. In response to completing the workflow process for a particular label request, the system can submit the resultant workflow documents and label request 106 to the health authority submission 116.

For example, during a workflow step, Subject Matter Experts (SMEs) are called in to review and later approve the veracity of the content, clinical trials documents that may be attached documenting possible drug interactions, side effect, etc. Authors can be tasked with writing and editing different sections of the label document and taking in the annotations and comments from reviewers. The management server 110 can track versions of the document associated with an Audit Trail recording to identify who made changes and when. Additionally, the management server 110 can track and record meetings associated with the workflow processes, and the management server 110 acquires each of the external interactions that typically happen over email or in face-to-face meetings.

The health authority submission 116 can include one or more processes related to submitting workflow documents and data from the label request 106 to a health authority. The health authority can include a health authority agency that can provide information about industry changes, safety warnings, and ensures legal compliance and quality services related to country or region regulations. Additionally, the health authorities can establish rules, regulations, and accreditation relating to medical products, devices, and pharmaceutical drugs subject to regulations. The management server 110 can package together the data from the label request 106 and the data resulted from the workflow process and submit this data to the health authority for approval in the health authority submission 116. The management server 110 can submit the new or updated label to Health Authorities according to a regulated and structured format involving a folder structure where the documents are provided for their consumption. The management server 110 can support and streamline this process. Examples of a health authority can include, for example, the Food & Drug Administration (FDA), the Agency for Healthcare Research & Quality (AHRQ), the Centers for Medicare & Medicaid Services (CMS), and the Centers for Disease Control & Prevention (CDC). Other examples are also possible.

In response to submitting the data to the health authority, the management server 110 can receive data from the health authority relating to the label request. In some examples, the management server 110 can receive data from the health authority relating to the label request through one or more APIs directly connected to the health authority. In some examples, the management server 110 can receive data from the health authority relating to the label request through email, mail, or other formats. The data from the health authority can indicate whether the health authority approves/denies the label request associated with the label request 106 and provide data information that describes how the submitting entity, e.g., user 102, can make corrections. In response to receiving approval from the health authority, the management server 110 can initiate the processes related to implementing, e.g., activating the commercialization process in a given country, the label request 106. Implementing the label request 106 can include, for example, activating the commercialization process in a given country and a process of distribution.

In some implementations, the processes related to implementing the label request 106 can include label content localization 118, artwork management 120, and dashboard 122. The label content localization 118 can include the creation and translation of the label depending on the different countries. For example, the label request 106 can indicate one or more locations where the product is intended to be sold. The management server 110 can rely on the label content localization 118 to create a label for the product and to translate a language of the corresponding label to a language used where the product is to be sold. The label content localization 118 can generate the contents of the label using the data included in the templates created from the tasks during the workflow processes 114.

In some implementations, the artwork management 120 can include one or more modules related to a creation of artwork associated with label. Each label generated by the management server 110 can include data from the supporting documents and artwork that are attached to the label. The artwork can be a brand image associated with the product that is specifically designed to represent the product or a modified image produced from a hand drawn image from the user 102 and includes required information as approved by a regulating health authority. In some examples, the branding aspect can be optional media for the product. The artwork management 120 can generate an image for the label and incorporate the image on the label using instructions from the label request 106. Additionally, the artwork for the label can encompass the layout of the label that includes the image.

In some implementations, the artwork management 120 can include a blockchain for securely managing the content of the label. A blockchain can include processes for distributed management of transactions while at the same time maintaining trust and distributed trust in those transactions. The blockchain can include a list of records, or blocks, which are linked together. Each block can include a pointer as a link to a previous block. Multiple parties can record transactions in the blockchain and/or verify previous transactions performed by others. The blockchain can also be referred to as a distributed ledger.

In a blockchain environment, blocks, which can hold data, are chained together by storing information in a block, which indicates the preceding block. A block can further include timestamp information and validation information. In this sense, a history of the data being stored in the blockchain can be accessed by moving along the blocks of the blockchain. Moreover, a blockchain can be held privately, e.g., in a centralized manner, or held publicly in a less centralized manner.

In the smart label management system 100, the artwork management 120 can include a blockchain that stores artwork related to labels, e.g., label content. The blockchain can include, in each block, a timestamp, verification information, and information related to a particular version of artwork for a label. Each connected block in the blockchain can include different versions of artwork for a label. For example, block 1 can include version 1 of artwork for label A, block 2 can include version 2 of artwork for label A and is connected to block 1, and block 3 can include version 3 of artwork label A and is connected to block 2. In this example, version 1 is older than version 2 and version 2 is older than version 3. In this manner, the artwork management 120 can view different versions of artwork for a particular label. Similarly, the artwork management 120 can include multiple blockchain environments for each label of artwork that is created. In some cases, the labels can be minted as non-fungible tokens (NFTs).

The artwork stored in the blockchain can include artwork approved by the health authorities, artwork approved by the management server 110, artwork provided in the label request 106, and artwork produced for distribution of the label. Moreover, the artwork stored in the block can act as a validation of the truth. In some implementations, artwork for a label may undergo various tweaks and edits. User 102, an automated artwork engine, or an external regulatory system can perform these edits, to name some examples. Each time new artwork is generated, artwork is changed or edited, the management server 110 stores the revised artwork for a particular label in a block on the blockchain. That way, at a later point in time, when artwork is being manufactured and preparing to be processed through a supplier chain, the management server 110 can ensure the artwork for a label is the correct artwork by verifying against the artwork in the latest block in the blockchain. By storing the artwork for a label on the blockchain, the management server 110 can ensure an immutable source of truth exists for artwork related to a label request.

In some cases, the smart label management system 100 can store approved local label content in a copy sheet. The copy sheet can combine package product information such as a global trade item number (GTIN) that are stock-keeping unit (SKU) specific. A copy sheet can include a 1-1 relationship to a packaging artwork where a summary of product characteristics (SMPC) could support various copy sheets. The final label and copy sheet for a label can be submitted to the block chain to reduce errors when content is being re-used outside of the labeling process and to facilitate validation between any artwork and the approved copy sheet.

In some implementations, the dashboard 122 can include one or more GUIs that display active status information relating to a label request. Specifically, the dashboard 122 can include one or more GUIs to be displayed on an external display connected to the management server 110. Additionally, the management server 110 can transmit the one or more GUIs of the dashboard 122 to client devices, e.g., client device 104, which connect to the management server 110 over network 108. For example, the application launched on client device 104 for providing the label request 106 can also display the dashboard 122 to user 102.

The dashboard 122 can display information relating to the initial creation of the label request to the submission of the request to the health authority. Specifically, the dashboard 122 can display active status information for documents, workflows, tasks, and user actions for the end-to-end experience of the label request to the health authority. The dashboard 122 can guide the viewing user 102 to various options during the end-to-end experience and anticipate next actions during the process of the label creation. For example, the actions on workflows can include edit, put on hold, withdraw, and view details. The actions can include start a new request and filter by at risk, overdue, on track, assigned to a user, checked out by a user, and completed, to name a few examples. The dashboard 122 can customize filters according to the user profile. In some cases, viewers of the dashboard 122 may not be able to see workflows or take action such workflows. Rather, the viewers can see their favorited documents and access these documents in view only mode. The key profiles of the dashboard can include the GLL, the LLL, a document coordinator that manages workflows, authors that edit documents assigned to them, and subject matter experts can review and annotate label documents.

The dashboard 122 provides various advantages over existing dashboards and user interfaces. Specifically, the dashboard 122 provides automation and digitization of extensively manual processes performed when creating a label. Moreover, the dashboard 122 enables productivity to be increased by supporting easy tracking of the label creation process and enabling users to act while viewing the active status information on a single display. Additionally, by providing status updates and active task information on a dashboard 122, the smart label management system 100 can reduce task complexity and time spent by the user 102 to sort through the various processes and documents related to label creation. Illustrative examples and descriptions related to the dashboard 122 will be described below.

In some implementations, the management server 110 can provide status updates 124 to the client device 104. The status updates 124 can include active status information provided from the dashboard 122. Similarly, the status updates 124 can include updates related to results from the approval of the label from the management server 110 and results from submission of the label to the health authority. The management server 110 can provide these results to the client device 104 (i) on a periodic basis, (ii) when the results are generated or received by the management server 110, and (iii) based on a status request transmitted by the client device 104.

FIG. 1B is a block diagram that illustrates an example of modules 101 for processing a label request of a smart label management system. The modules 101 illustrate the modules, processes, and algorithms used to perform the functions related to the label request 112 from FIG. 1A. These modules 101 can be automated processes or processes performed by individuals or parties external to the management server 110. The modules 101 can include receive label request 126, submit to impact assessors (IA) 128, IA review the label request 130, update status request 132, confirmation task started 134, approve request 136, and send to workflow process 138. The modules 126 through 138 can be performed in the processes illustrated, in a different process, and with additional or fewer processes.

During 126, the management server 110 can receive the label request. Specifically, the management server 110 can receive the label request, e.g., label request 106, from a particular client device. The management server 110 can receive multiple label requests at a time from different client devices. In some implementations, a client device may submit multiple label requests to the management server 110. The management server 110 can process each of the received label requests in parallel or in serial in the order in which they were received.

The management server 110 can analyze the received label request and determine a recognized user submitted the label request using the authentication information provided in the label request. Once authorized, the management server can analyze the received label request to determine information pertaining to the type of label that is desired to be approved and created. For example, the label request may include information related to development and testing of a product, e.g., drug, medical device, etc., associated with the label, symptoms of the drug, side effect, compound plans, clinical trial results, efficacy study results, expected therapeutic area (TA), target plan, safety test results, government entries, data relevant to eCTD regulatory filing, data regarding which personnel were involved in the design manufacture, approval, sale of the drug, and other information. The management server 110 can extract this information from the label request and store this extracted information in memory.

During 128, the management server 110 can provide the extracted information from the label request to impact assessors (IAs). The impact assessors can be external processes, users, or departments that perform various functions. These functions can include, for example, considering the implications, for people and their environment, of proposed actions while there is an opportunity to modify or abandon the proposals associated with the extracted information.

Specifically, in the example of a drug label request, the impact assessors can determine the impact of the changes to an existing label document and estimate if the information provided in the request is sufficient to make the change in the time allocated to such changes. If the drug label request is a new label, e.g., if the management server 110 determines the drug label request corresponds to a new label, the impact assessors can also assess if the information is sufficient.

During 130, the impact assessors can review the extracted information provided with the label request. As mentioned above, the impact assessors can determine the impact of the changes to an existing label document and estimate if the information provided in the request is sufficient to make the change in the time allocated to such changes. In some examples, the assessment can take less than a week to complete. In some examples, the assessment can take longer than a week to complete.

During 132, the management server 110 can update the status of the label request. Specifically, the management server 110 can transmit a periodic notification of status to the client device that submitted the label request. The status can indicate, for example, that the label request was submitted to impact assessors, that the impact assessors are reviewing the label request, that the impact assessors have either approved or denied the label request, that the label request has been approved and is now being sent to workflow processes. Other examples are also possible.

In some implementations, the management server 110 can display the status updates to the dashboard 122 for the user review. A user who submitted the label request can access the dashboard 122 of the management server 110 and monitor these status updates through the dashboard 122. The dashboard 122 can display the status updates and one or more changes to the status updates in real time. In this manner, a user that submitted the label request can monitor processes related to their label creation.

During 134, the management server 110 can receive confirmation from the impact assessors that the label request has been approved. This information can include a detailed outline describing how the impact assessors believe the drug associated with the label request will have an impact on socioeconomic factors. The management server 110 can save this information from the impact assessors in memory and provide this information in a report or in an audit report to the user that submitted the label request at a later point in time.

During 136, the management server 110 can provide a confirmation to the user through their respective client device or through the dashboard 122 indicating that the impact assessors have approved the label request and processing of the label request has initiated. As previously mentioned, the processing of the label request by the management server can include designating one or more tasks to assignees. The user may interact with the dashboard 122 to determine the next processes in the processing of the label request and an amount of time predicted for the processing of the label request to be completed. The dashboard 122 may provide, for example, a predicted timeline of events for the completion of the label request processing and an amount of time required for the label request processing to be completed. In some examples, the management server 110 can provide a detailed outline of the next processes in the processing of the label request and the amount of time predicted for the processing of the label request to be completed to the client device 104 of the user that submitted the request.

During 138, the management server 110 can transmit the extracted information from the label request and the detailed outline from the impact assessors to the workflow processes. The workflow processes can utilize this information to initiate workflows for creating documents to eventually be submitted to the health authorities. In some cases, the management server 110 can trigger a workflow automatically. The automatic triggering of a workflow, and trigger a multi-workflow process with packet segregation. This automatic triggering may require human intervention from a document coordinator. A workflow can correspond to processes related to managing document creation and document updates. For example, the different types of workflows can include authoring a document, reviewing a document, approving of a document, managing translations, managing artwork, and managing submissions. Additionally, the dashboard 122 can illustrate status updates regarding the various workflows being performed for the label request or documents that are not part of the label request.

FIG. 1C is a flow diagram that illustrates an example of workflow process 114 of a smart label management system. In some implementations, the workflow processes 114 of FIG. 1C illustrates the processes performed during the workflow process 114 from FIG. 1A. Specifically, the workflow process 114 can include various functions performed that relate to the approval of the label request. For example, the workflow process 114 can perform processes related to managing, reviewing, and approving different documents related to the label request. This can include different types of workflows, e.g., authoring a document, reviewing a document, and approving a document

In some cases, a document or label creation/update life cycle can be supported with a multi-workflow process or a series of single workflow processes. In a multi-workflow process, a user or machine can assign a particular document cycle to be completed in N number of iterations. These iterations can include N number of times of looping over authoring, reviewing, and approving various tasks. A cycle may be supported by a multi-workflow process based on a complexity of the overall work being performed on a set of documents. In some cases, the cycle can be linked to a label request. In some cases, the cycle may not be linked to a label request but can support non-label related document processing that also perform a validation, submission, and distribution process. For example, a more complex process requires more than N number of cycles to be completed. In a single workflow process, a user or machine can assign a particular workflow process to be completed in 1 cycle. In the single workflow process, this can involve more than one user assigned to individual tasks. This cycle can include one process of authoring, reviewing, and approving a task.

In some cases, if one of the workflow processes fails, the management server 110 can return the document back to the draft stage. Here, the management server 110 can require the document to be re-worked until approval is reached. Each failure of a document can trigger a new cycle of authoring, reviewing, and approving.

During 140, the workflow process 114 in the management server 110 receives data indicating the impact assessors have approved a new label request. The workflow process 114 can receive this data from the label request 112 module. Then, in 141, the workflow process 114 can assign a document coordinator to this workflow and notify this document coordinator to initiate the workflow processes. A document coordinator can manage the various workflows in a document management system. Specifically, a document coordinator can setup workflows and track workflow statuses of each assignee in the workflow of the document management system. Additionally, the document coordinator can designate durations for the completion of each task. The different tasks designated by the document coordinator for a workflow can relate to verifying the contents of the label request such as, for example, producing contents of a product associated with the label, and verifying the contents of a product associated with the label, among others. In some examples, the tasks can include verifying the number of refills included in the label creation request is safe for the patient's use and medically intended as prescribed by the prescriber.

A document coordinator, which can be a person or an artificial intelligence product, can instruct one or more assignees to create one or more templates using information from the label request, review data filled in the templates using information from the label request 106, and approve data filled in the templates using information from the label request 106. Other tasks of a workflow can include assigning tasks to assignees, authoring a workflow packet of various templates, reviewing workflow packets, reviewing assigned tasks, rejecting document workflows, and approving workflows. Moreover, the document coordinator can select a document type for the workflow tasks, country or region for the label request, select a language for the workflow task, and others.

During 142, the document coordinator can decide whether a process of creating or updating a document, which can be a part of a label request package, should be performed in a multi-workflow process 143 or a single workflow process. The document coordinator may decide whether to use either a multi-workflow process 143 or a single workflow process based on a complexity of the document update/creation. For example, if a task corresponds to filling in a template document using drug composition, drug symptoms, side effects, compound plans, clinical trial results, and efficacy study results, then the document coordinator may assign the filling in of the template to single workflow process because the complexity of this task is deemed to be low. In some cases, the document coordinator can fill out the template and create the document as a placeholder. The workflow can be setup and attached to either the placeholder for a new document or a previous version of a document. Document packets can be attached to a workflow to be assigned to a group of authors who can either claim the task individually or work collaboratively. On the other hand, if a task corresponds to translating a chemical composition of the drug to a different language for the label, e.g., from English to French, then the document coordinator may assign the complexity of this task to be high due to the various nuances of language translation. In response to assigning a task to have high complexity, the document coordinator can assign this task to be performed in a multi-workflow process to ensure the workflow task is performed over multiple iterations to ensure optimal translation from English to French.

In some implementations, the workflow processes 114 can include one or more trained machine-learning models that can predict a likelihood of whether a task has high complexity or low complexity. The one or more trained machine-learning models can receive, as input, data specifying the workflow, a number of tasks assigned for the workflow, data specifying types of tasks to be performed for the workflow, a number of assignees assigned to each of the tasks, a length of time that each assignee has for completing each of the tasks, and an estimated length of time for completing the workflow. In some cases, the one or more trained machine-learning models can produce document updates that can be linked and automated using the above inputs. For example, the one or more trained machine-learning models can include a lightweight convolutional neural network (CNN) for predicting task complexity for a workflow.

In response, the one or more trained machine-learning models can output a likelihood or probability that a task is of high or low complexity. In response, the workflow processes 114 can compare the output likelihood to a threshold value to augment the prediction capability of the trained machine-learning models. For example, the threshold value may be set to 70%. If the trained machine-learning models output a 71% likelihood that a task is complex, then the workflow processes 114 can compare the 71% likelihood to a threshold value of 70% to deem the task as a complex task. In this example, the output likelihood satisfies the threshold value, e.g., is equal to or greater then. However, if the output likelihood is less than the threshold value, e.g., does not satisfy the threshold value, then the workflow processes 114 can deem the task as a low complexity task. In some cases, the trained machine-learning models can detect how many authors, reviewers, and approvers should work on a workflow. In some cases, the trained machine-learning models can identify which of the authors, reviewers, and approvers are available and which assignee should be associated with particular tasks of a workflow. The trained machine-learning models can detect users and identify which assignee should be assigned to the particular tasks of a workflow based on workload estimates, document size, which SMEs are best for the domain, and a type of product. In some cases, the componentization of the documents can also be automated.

The management server 110 can train one or more machine-learning models based on prior determinations of tasks being of high or low complexity. For example, the management server 110 can train the one or more machine-learning models using information that designated a task. If a document coordinator initially designates a task as a single workflow process, e.g., a low complexity, but in reality, the task requires multiple iterations, then the management server 110 can use the data indicative of the workflow, e.g., label request information, tasks to be completed, designation of the workflow as high or low complexity by a document coordinator, and number of cycles to complete the task, as training data. The training data can include areas of expertise and history of reviewers/approver assigned to specific types of product, for example. The expertise can be in specific regulatory issues for a region, type of product, medical area, such as, cancer treatment, lung disease, COVID detection, and other, to name a few examples.

In this case, the trained machine-learning model can predict a likelihood of a task complexity, an expertise area, and/or a language using the aforementioned factors. For example, if the workflow coordinator designates a task to be a single workflow process, but the number of cycles performed for this task is ten, then the trained machine-learning model can recognize a mistake because this task should have been designated as a multi-workflow process. In these types of situations, the trained machine-learning model can catch and produce accurate likelihoods for tasks that may deem to be performed in a single workflow but in fact should be performed in a multi-workflow environment. The alternative to this case is also true, e.g., tasks that were deemed for multi-workflow but should have been performed in a single workflow. The management server 110 can train the machine-learning models using stochastic gradient descent, Bayesian optimization, regression, clustering, and multi-label optimization, to name a few examples.

Similarly, the management server 110 can provide feedback to the trained machine-learning models during their implementation of producing task complexities, domain expertise, and geographical relevance. For example, the trained machine-learning model can output an indication that a task is highly complex, e.g., converting a document's language from English to Spanish, and during the actual processing of the tasks in a multi-workflow sense, the multi-workflow task is completed in one cycle. If this is the case, then the management server 110 can provide feedback data to retrain the trained machine-learning model. The retraining can be performed to ensure the next time the trained machine-learning model sees similar input that the output of low complexity is produced. The feedback data can include the previous input data used to produce the output that the task is highly complex and data indicating the output should be low complexity, to ensure the trained machine-learning model outputs the correct complexities for future similar workflow tasks. In this case, the management server 110 can force the trained machine-learning models output to be of low complexity the next time similar input data is received. The opposite is also true, e.g., a task was deemed to be of low complexity by the trained machine-learning model but in actuality, the task required multiple cycles to complete. In some cases, another area of training can include anticipating packet segregation decisions to decrease over time due to manual intervention by the document coordinator.

During 143, the document coordinator can decide that a particular document process should be performed using a multi-workflow iteration. Moreover, the document coordinator can assign one or more tasks of the workflow to be performed by one or more assignees in a predetermined time period. These one or more assignees can perform at least one of the authoring, reviewing, and approval tasks. For example, the document coordinator may assign a first assignee to perform the translation of a document from English to French in no more than three days, the second assignee to reviewing the translation of the document from English to French in no more than two days, and the third assignee to approve the translation of the document from English to French in no more than two days. Other examples are also possible.

Continuing with the above translation example, after the first assignee authors the translated document, e.g., completes the translation, the second assignee can review the translation document and determine whether the translation is proper. In some implementations, the first assignee can provide data to the management server 110 to indicate the translation is complete. The data can be, for example, an email, a notification, a text message, or other, to indicate the translation is complete. Once the management server 110 receives the notification, the document coordinator is automatically notified. If the first assignee cannot complete the assigned task within the predetermined time, then the first assignee can notify an application of the management server 110 to signal to the document coordinator that more time is requested to complete the task. However, if the first assignee can complete the assigned task within the predetermined time, then the first assignee can complete the task and submit the completed task to the application of the management server 110. In some cases, the multi-workflow system of the management server 110 can automatically send the translated document to the second assignee. In some cases, the document coordinator can manually create a workflow that contains the individual tasks, assignees, and assigned documents. Generally, the documents in the management server 110 can be attached to the task assigned to the next assignee in line and the document coordinator is notified automatically that the authoring of the workflow task has been completed when all assignees have marked their tasks as finished.

The content of a document is validated through the review and approval workflows. At the end of such workflows, the document or packet of documents is marked as approved or rejected. In some cases, a packet can be partially approved. At this stage, a document is either approved or reject. The document coordinator can decide whether the failed task requires a low complexity fix or a high complexity fix. If the document coordinator decides that the failed task requires a low complexity fix, e.g., a small number of errors need to be changed, then in 146, the document coordinator can perform a quick review of the rejected document, and in some cases, perform changes themselves, and send the document directly into a new approval flow. In this case, the document coordinator performs the changes themselves, the review workflow steps can be skipped. In 148, the system can increment the counter. Generally, each cycle counter for a workflow task starts at a value of zero and increments by a value of one for each iteration. This process repeats until the workflow iterations are complete, e.g., all users marked their task as complete, and the document is approved in a major version. The minor versions are intermediate iterations on the document during the authoring, review, and approval cycle.

If the document coordinator decides that the rejected document(s) require a high complexity fix, e.g., a complete revision of the solution, then in 147, the document coordinator can update the workflow parameters to decide whether the workflow task should be assigned to one or more different assignees or requires multiple workflows. The document coordinate may choose collaboration, e.g., all authored work on the document, or a first assignee to claim does the work and others are cancelled. A workflow may include multiple steps and assignees can be selected for each of the steps.

In some implementations, the document coordinator can decide whether a given workflow should be skipped in 149. For example, if a task is too complicated, if an assignee does not yet have the requisite material required for completing the task, or if a workflow is to be put on hold, then the document coordinator may decide whether to wait to perform the task at a later time. In some cases, the document coordinator may transmit a request for information to the client device of the user that submitted the request. In this case, the document coordinator can place a task or workflow on hold until the user that submitted the request provides the requisite material for a particular workflow to be completed responsive to the additional request.

Continuing with the above example, if the review and approval workflow determines that the translation is approved, then the translation can move to the next step and an indication that the translation was approved is recorded by the system. In response, the document coordinator and the GLL are notified. For local labels, the LLL is notified.

In some implementations, if the document coordinator or the trained machine-learning model indicates that the task is of low complexity, then the workflow task can be deemed to be performed by a single workflow task. In the single workflow task, a first set of assignee(s) can be assigned to author a document in a predetermined amount of time in 150, a second set of assignees can be assigned to review the authored document in a predetermined amount of time in 151, and a third set of assignees can be assigned to approve the reviewed document(s) in a predetermined amount of time in 152. Due dates are specified for each workflow. In some cases, a workflow may be broken down into steps each with a due date. Similarly, a single assignee can be assigned to perform each of the tasks in 150, 151, and 152.

After the task for the single workflow process has passed through the authoring, reviewing, and approval stages of 150, 151, and 152, respectively, then the document coordinator is notified and decides how to proceed in 145. Specifically, the document coordinator can decide whether to complete the workflow task, perform a quick review and confirm in 146, or update workflow parameters in 147. If either 146 or 147 are performed after the single workflow process, then the document coordinator can determine that this workflow task should have been designed a workflow multi-task and can provide feedback information to update the trained machine-learning model. In some examples, the document coordinator can decide whether to complete the single workflow task or whether to perform 146 or 147 based on whether the approved task from 152 is free of errors, includes complete and accurate information from a regulatory standpoint, has the proper functions performed, or any other designation required for a completed task. If the document coordinator decides that the approved single workflow task requires updates or different tasks to be performed, then the document coordinator can designate tasks related to 146 or 147 to be performed. This process can then repeat until the assignees have approved the document(s).

In some implementations, during the workflow process 114, a chatter tool can be provided between the assignees and the document coordinator. The chatter tool can enable different assignees to communicate about the documents. Additionally, the chatter tool can enable the different assignees to communicate with the document coordinator regarding notifications of the task. In some examples, the chatter tool can be active when and while required for the life of a label request or a workflow and its processes.

In some implementations, the management server 110 can include one or more plugin application programmable interfaces (APIs). The plugin APIs can enable communication with different third party planning and tracking systems. These plugin APIs can be implemented by stage gates. Stage gates can include plug and play ready APIs that enable tracking for which the system and its users have no deliverables. However, for the next stage, labeling has a large number of deliverables to complete within a given time window. As such, status updates of labels included within a submission, trigger tasks, and notify others of tasks.

A stage gate can define a stage name, date, and whether a label has a deliverable. When a gate is tagged as a labeling deliverable stage, tasks and impacted labels are activated. For example, at the end of a horizontal bar of a Gantt chart, a stage gate can be defined that indicates whether a deliverable is required. A deliverable may be, for example, an authored workflow task to be complete and provided back to the document coordinator.

FIGS. 1D and 1E are additional flow diagrams that illustrate examples of workflow processes of a smart label management system. Specifically, FIG. 1E is a continuation of the processes performed in FIG. 1D. In some implementations, the workflow processes 114 of FIGS. 1D and 1E use packet segregation for label request processing. Specifically, the workflow processes 114 of FIGS. 1D and 1E perform a workflow process when a task relates to a document. Additionally, the workflow processes 114 of FIGS. 1D and 1E are similar to the workflow processes 114 performed in FIG. 1C.

During 153, a setup stages/tasks are performed. In response to receiving notice that the label request has been approved for processing, the workflow process 114 of the management server 110 defines one or more tasks to be performed. These tasks can include, for example, authoring a packet workflow, reviewing packet workflow, reviewing task assigned, rejecting document workflows, and approving document workflows, to name a few examples. 153 is similar to 140 of FIG. 1C.

During 154, the tasks of a workflow are assigned to a document coordinator. The management server 110 assigns a document coordinator to these tasks of a workflow and notifies the document coordinator to initiate the workflow process. During 155, the management server 110 assigns a workflow template. A workflow template can be assigned as a dependency of a document record type. For example, a workflow template associated with the document type SmPC may require for two stages of review to take place before reaching an internally approved state. In some examples, a workflow template may include a translation or back translation stage for a document type. The workflow template can include one or more required fields relevant to the type of the label request that is to be reviewed by the health regulatory authority.

During 154 and 155, the document coordinator initiates the authoring workflow for a packet of documents. Specifically, the document coordinator can decide whether this label request should be performed in a multi-workflow process or in a single workflow process based on a complexity of the task or other criteria. In some cases, the document coordinator can utilize a trained machine-learning model to augment the decision making of whether a task involves high complexity or low complexity. In response to determining whether the process is a multi-workflow or a single workflow, the document coordinator can assign workflow tasks to one or more assignees and due dates for completions of the overall workflow. In some cases, the document coordinator can assign workflow tasks for each step in a workflow if broken down into steps. Each of the assignees can perform a respective task assigned to them in a given workflow, e.g., authoring, reviewing, or approving. 154 and 155 is similar to 143, 149, 150, and 151 of FIG. 1C.

During 158 and 159, the assignees can review the document(s) and finish the review of the document(s). In 159, the assignees can mark their task as “finished” once completed. The assignees can also complete their assigned tasks during 158 and 159. These tasks may take a set amount of time to complete, for example, an hour, a day, three days, a week, or a different time frame. After a task has been completed or a task not passed one of the assignees review or approval, then during 160, the results of the task are passed back to the document coordinator.

During 161, the document coordinator can decide whether to segregate a document packet or not: if segregated, the approved document(s) moves to the next step in their cycle and the rejected ones require another round of authoring, review, and approval. If segregation is not possible for the workflow used, then the packet is fully rejected if a subset of documents is rejected. This complex process involves many processes and can provide data indicative of the processes to an interface. If the workflow is set to automate this decision point, then an autonomous process can determine whether the document packet is segregated or not. The autonomous process, which can be a trained machine-learning model, can replace the document coordinator at any time during the workflow processes 114. 161 is similar to 145 of FIG. 1C.

During 162, the document coordinator can decide to reject the entire document packet due to error, incomplete tasks, or other. Then, during 163, the document coordinator can decide to rerun the workflow with different parameters. 163 is similar to 147 of FIG. 1C. After updating the workflow parameters, the management server 110 can update the cycle counter by a value of one in 164. 164 is similar to 148 of FIG. 1C. In response to updating the cycle counter, the management server 110 can move the process to 156 for authoring of the packet workflow with the updated parameters from 163.

In some cases, the document coordinator can decide to segregate a document packet. Then, during 165 in FIG. 1D, the document coordinator can decide whether to rerun the workflow with different parameters in 166 if new processes need to be performed or whether the workflow approval is complete in 168. If the document coordinator decides to rerun the workflow with different parameters in 166, then the workflow task can updated with different parameters, and then in 167, the management server can update the cycle counter by a value of one. This process continuously repeats until the workflow approval is complete in 168.

During the workflow processes of 114 in FIGS. 1D and 1E, the documents can be maintained in different states. For example, the states can include in authoring, in review, on hold, draft, rejected, approved, in approval, and reviewed, among others. Each of these states can be monitored by the document coordinator and displayed on the dashboard 122 for the user that submitted the request. Additionally, these states can be submitted as status updates to the client device of the user that submitted the label request. In some cases, the documents can proceed to a translation workflow.

FIG. 1F is a block diagram that illustrates an example of modules for health authority submission of a smart label management system. Specifically, FIG. 1F illustrates the modules and processes performed after the workflow approval process completes. When the workflow approval process completes, the management server 110 can provide a notification on the dashboard 122 for the user's review to indicate that the workflow process has been process and approved and the submission to the health authority is currently in progress. In this manner, the user that submitted the request can review the status of their label request in real time.

During 169, the health authority submission 116 receives data indicating that a label document has been approved from the workflow processes 114. The data can include the one or more label documents that were approved, data from the label request 106, and detailed information from the impact assessors. Additionally, the data received from the workflow processes 114 can include historical workflow data. For example, the historical workflow data can include a number of cycles for approving the documents of the workflow, iterations of various documents, revisions of different documents, a history of edits made to the documents by the document coordinator and the assignees, and other information. The health authority submission 116 can prepare and package this information to submit to the health authority.

During 170, the health authority submission 116 assembles a document packet of approved workflow data. Specifically, the health authority submission 116 packages the workflow documents that were approved, data extracted from the label request 106, and detailed information from the impact assessors. In some cases, the health authority submission 116 may generate a ZIP file or other digital grouping of these documents or a package of these documents to send in the mail. In some cases, the health authority submission 116 can generate approved labels for a submission coordinator process, plan, and submit to the health authority. Before being submitted to the health authority, the approved labels may be reviewed by an SME for accuracy. In some cases, the health authority submission 116 can prepare labels to be provided to the health authority and fed-back into labeling once a decision has been received from the health authority.

During 171, the health authority submission 116 can create a submission package. The submission package can be a package of the grouped documents that is prepared to be sent to the particular health authority. The health authority submission 116 can generate a submission package of these grouped documents in a manner specified by the particular health authority. The submission package can be ordered in a particular manner, prepared to be sent in a particular manner, e.g., email attachment, mail package, or another manner of submission.

During 172, the management server 110 can submit the submission package to the health authority. As mentioned, the health authority can include, for example, the FDA, the AHRQ, the MHRA, the EMA, and others. In some implementations, the label request 106 can include data specifying the particular health authority. In some implementations, the management server 110 can determine the particular health authority for submitting the label request information based on a type of the label request. In some implementations, an individual of the management server 110 can hand deliver the submission package to the corresponding health authority.

During 173, in response to submitting the submission package to the health authority, the management server 110 can provide a status update to the dashboard 122. The status update can indicate that a submission package for the label request 106 has been submitted to a designated or selected health authority. For example, the status update can designate a date/time the submission package was submitted to the health authority, data specifying the health authority, an estimated amount of time to receive an approval or denial from the health authority, and/or predictions indicating a likelihood of being accepted.

The management server 110 can determine the estimated amount of time to receive an approval or denial from the health authority by querying a website, server, or another computer system associated with the health authority. Typically, the health authority can provide time estimates to clients seeking regulatory approval for an expected response. The management server 110 can extract this time estimate from the health authority and provide this time estimate to the dashboard 122 for the user's review.

In some implementations, the management server 110 can predict an amount of time to receive a response from the health authority based on previous estimated times for the health authority. For example, the management server 110 can identify averages, maximums, minimums, and other prior times related to an amount of time a user can expect to receive a response from the health authority. In some examples, the management server 110 can display that the last five submissions to the EMA resulted in a response time of five days or less. Other examples relevant to the particular health authority are also possible. The management server 110 can also indicate on the dashboard 122 whether the estimated response time comes from the health authority or estimated based on the management server 110's own analysis from prior health authority submissions.

During 174, the management server 110 can receive an approval notification from the health authority. The approval notification can indicate that the label request is approved for distribution. In some implementations, the response from the health authority can be a denied request. When the request is denied, the health authority may provide reasons for denial.

During 175, the management server 110 can provide a status update to the dashboard 122. Specifically, in response to receiving the approval notification, the management server 110 can provide the status update to the dashboard 122 that indicates the approval notification from the health authority. In some implementations, the management server 110 can provide data indicative of a denied request when the health authority denies the label request. Specifically, the management server 110 can provide the denied request and reasons for denial to the dashboard 122 and/or to the client device of the user that submitted the request so that the user can revise the label request with data addressing the reasons for denial. At a later point in time, the user can submit the revised label request with the data addressing the reason for denial in hopes of receiving an approval from the health authority.

During 176, the management server 110 can proceed to implementation of the label request 106 based on an approval from the health authority. The implementation of the label request 106 can include the creation of the label and distribution of the label on the product. The implementation of the label request 106 can also include the insertion of artwork on the label and validation that the distributed label matches to the label design designated by the user that submitted the label request 106.

During the implementations of the label request 106, the management server 110 can execute the label content localization 118 and the artwork management 120. The label content localization 118 can include generating the label and manufacturing of the label. Additionally, the label content localization 118 can include translating a language of the generated label to a language where the product associated with the label is to be sold. Moreover, the label content localization 118 can include functions for edit, modify, or otherwise remove portions of the label.

The artwork management 120 can include generating the artwork for the label, securing the generated artwork in a block of the blockchain, and validating the artwork created during the manufacturing of the label to the artwork secured in the blockchain. The validating ensures the artwork that is being supplied to distributors properly matches to the intended artwork stored in the block of the blockchain.

FIG. 1G is a block diagram that illustrates an interface 177 for monitoring a status of a label request in a smart label management system. The interface 177 illustrates a process for document branching. Specifically, branching can be described as a process of duplicating an object, e.g., document, under version control. A user can modify each duplicated object thereafter separately and in parallel so that the objects become different. These objects can be operated on in different branches. The interface 177 is typically performed during the workflow process 114 and the label content localization 118.

The branches can include a parent branch, a child branch, and other branches, to name a few examples. The branches can also include a trunk, which is designated as a baseline version for the object. Development that occurs in the branches include development on projects, e.g., documents or software, that have yet to be official released or submitted to workflow processes 114 or to the health authority. Moreover, branching implies an ability to later merge or integrate changes back from one branch to another, e.g., from a child branch to a parent branch. Ultimately, the changes in a branch can be merged into the trunk for baseline development.

For example, interface 177 displays a branch 178 and a trunk branch 179 in the localization user interface. In the branch 178, the workflow processes 114 or the health authority may approve a document 180 and a document coordinator can merge document 180 into the trunk branch 179. Merging a document into a branch includes merging changes from the uploaded document into a document stored in the trunk branch 179. Shortly thereafter, the document coordinator may receive updates on the document 180 in the trunk branch 179. An assignee or a user that initiated the label request may perform these updates or changes, for example. However, portions of a document or an entire document that is locked due to editing by another user cannot be merged to other branches or receive merge changes until the document is unlocked.

Continuing with the above example—after the trunk branch 179 receives updates to document 180, either the workflow processes 114 or the health authority can subsequently approve the document 181 in branch 178. Document 181 may be a modified version of document 180. To ensure that the different branches maintain the changes from the approved version of the document 181 in branch 178 or another branch, the management server 110 or a document coordinator can merge the changes from the approved version of the document 181 in branch 178 to the various documents across different branches, e.g., trunk branch 179.

Generally, the management server 110 or the document coordinator can manage the changes applied to different documents across the different branches. These changes can include, for example, changes to document name, changes to content within the document, permissions related to the document, change of a document type, and changes related to author or ownership of a document. Additionally, the content in a document can be componentized, so each letter, word, sentence, or paragraph, can be merged into different documents when changes occur. These changes can be applied from one branch to the other branches.

In some implementations, the changes can be merged across different branches when assignees submit the document changes to the trunk branch or to different branches. Similarly, when the workflow processes 114 or the health authority approves a document on a particular branch, the management server 110 can recognize this trigger and automatically merge the changes from the approved document on a particular branch to one or more different branches in response to receiving the approval. For example, the management server 110 can apply the changes of document 181 to each of the other branches in response to receiving an approval from the workflow processes 114 or the health authority of document 181. In this manner, the documents in the other branches can build additional detail on-top of pre-approved documents. This helps further the likelihood that the modified documents will also be approved given the underlying approval of the documents before they were modified.

In some implementations, the branching can spawn additional functions to be performed. These functions can include automatic language detection and automatic language translation. For example, if branch 178 includes documents that are written in English and trunk branch 179 includes documents that are written in French, then the management server 110 can ensure continuity exists when performing document merging. If the management server recognizes that document 180 is to be merged into the trunk branch 179, then the management server can first detect a language of the document 180. In response to detecting the language of document 180, the management server can detect a language of the documents in the branch that is to receive the merged document. For example, the language of the documents in the branch that is to receive the merged document, e.g., trunk branch 179, is French. The management server can detect the language differences between the documents in branch 178 and trunk branch 179 and perform automatic language translation to ensure the merge changes are continuous across branches of different languages.

For example, the management server 110 can automatically convert the English language of document 180 to the French language. After performing the language conversion of document 180, the management server 110 can validate the language conversion was properly performed. For example, the management server 110 may rely on one or more third party programs to verify the accuracy of the language conversion. After validating the language conversion was properly performed, then the management server 110 can merge the changes of the document 180 in French into the trunk branch 179. Thus, any changes of document 180 that were performed in English in the branch 178 can be properly saved in the trunk branch 179 after the merging the changes of the document 180 in the French language. Similarly, the management server 110 can automatically translate changes from documents that are pushed from an origin branch to other destination branches where the languages of the destination branches are different from the language of the origin branch. Other language examples are also possible.

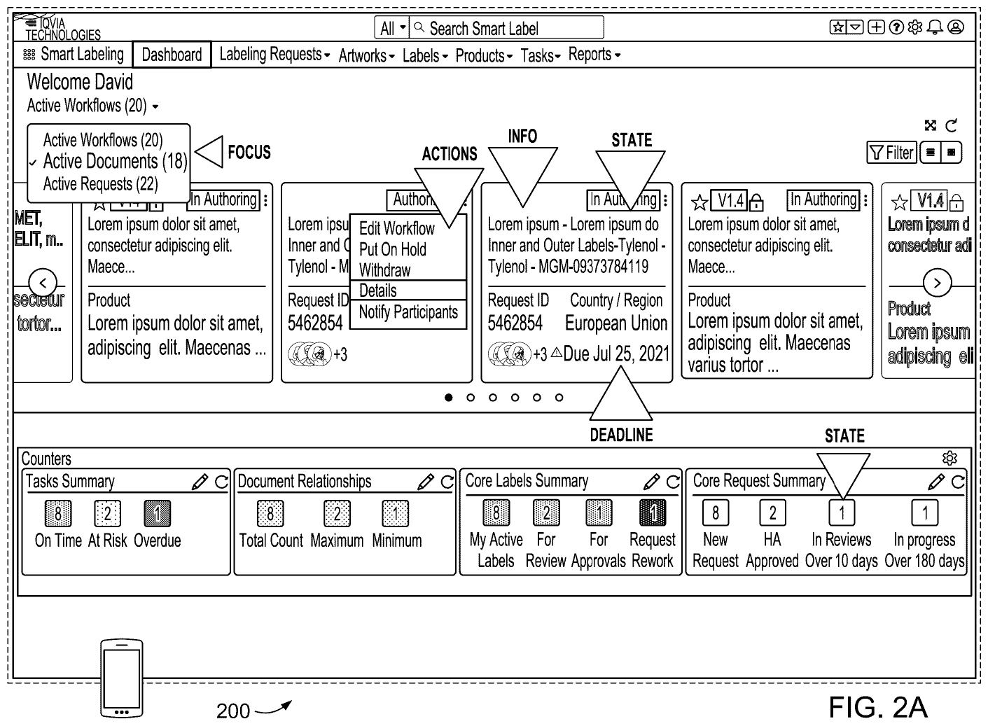

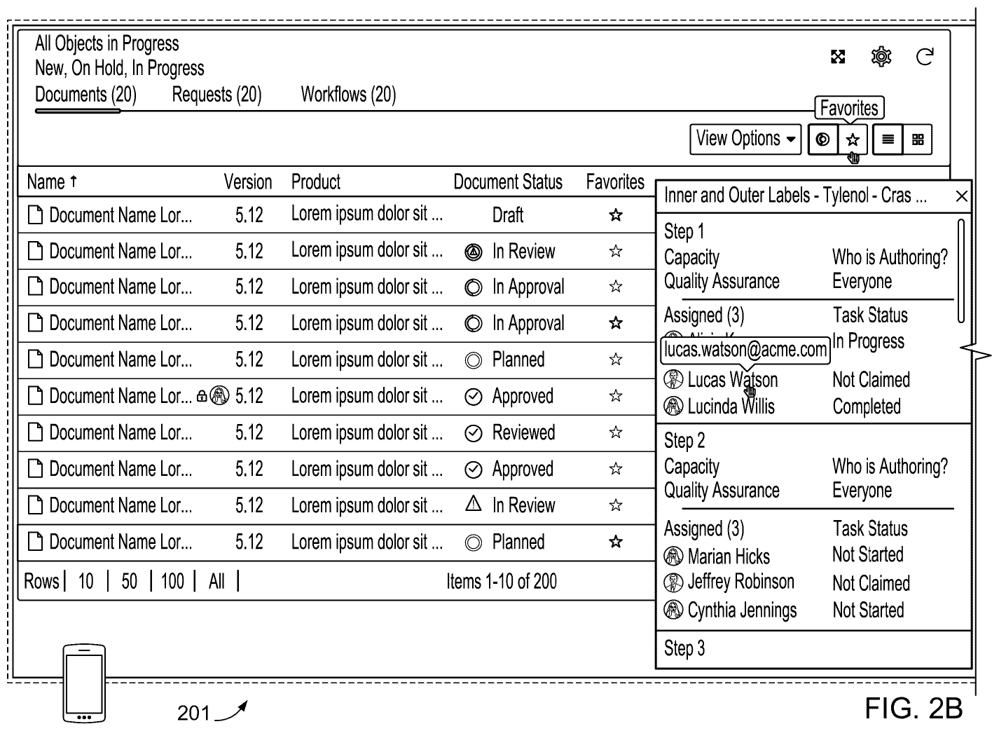

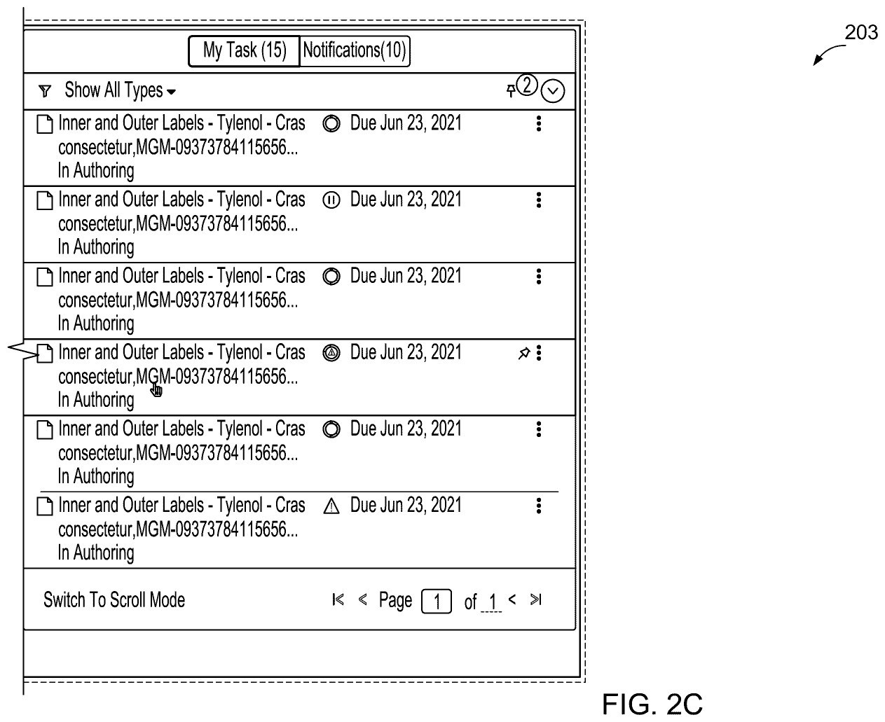

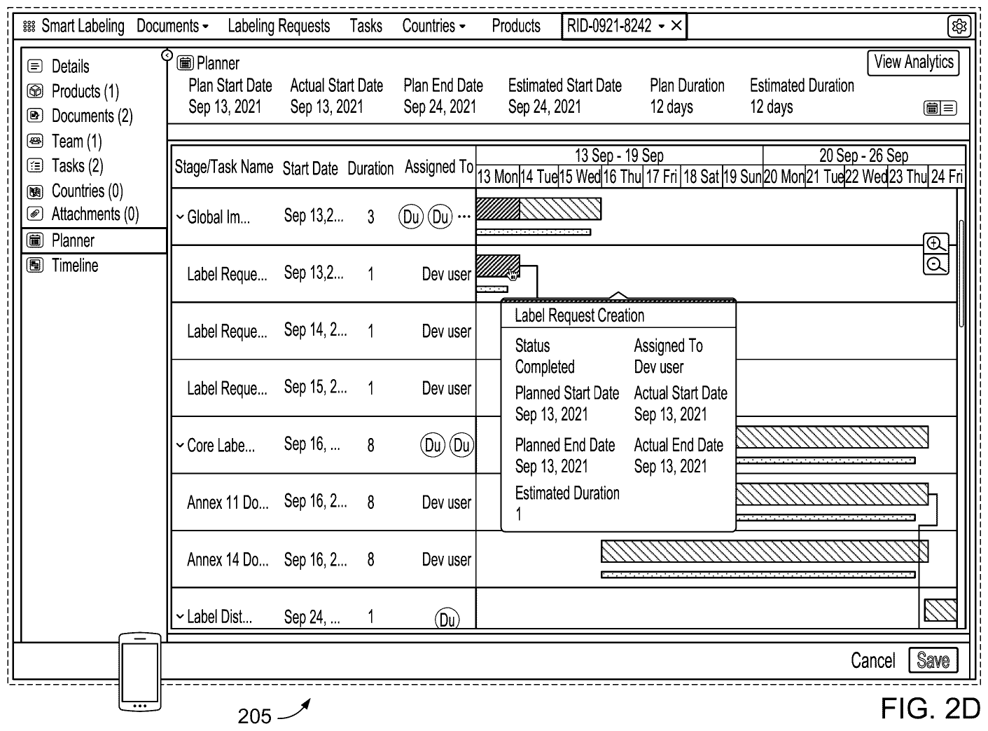

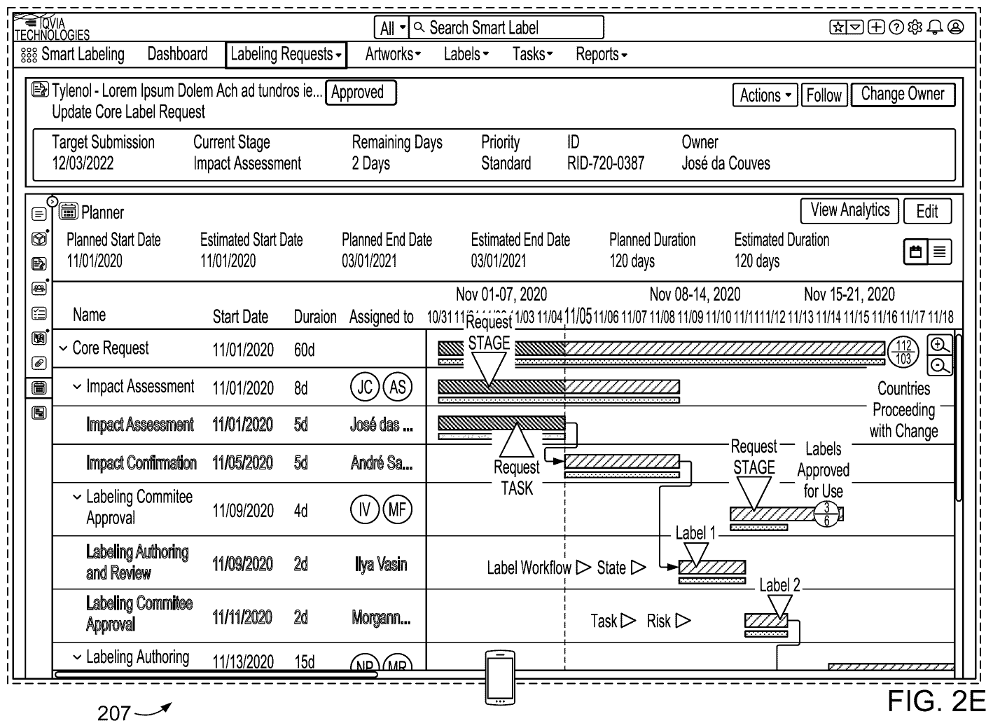

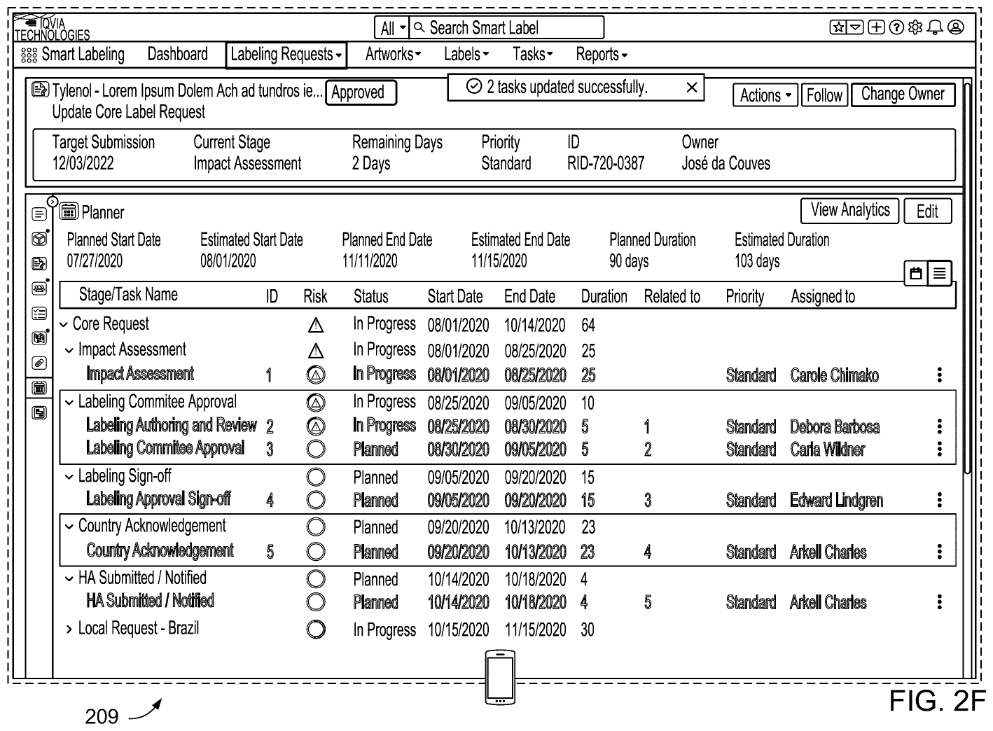

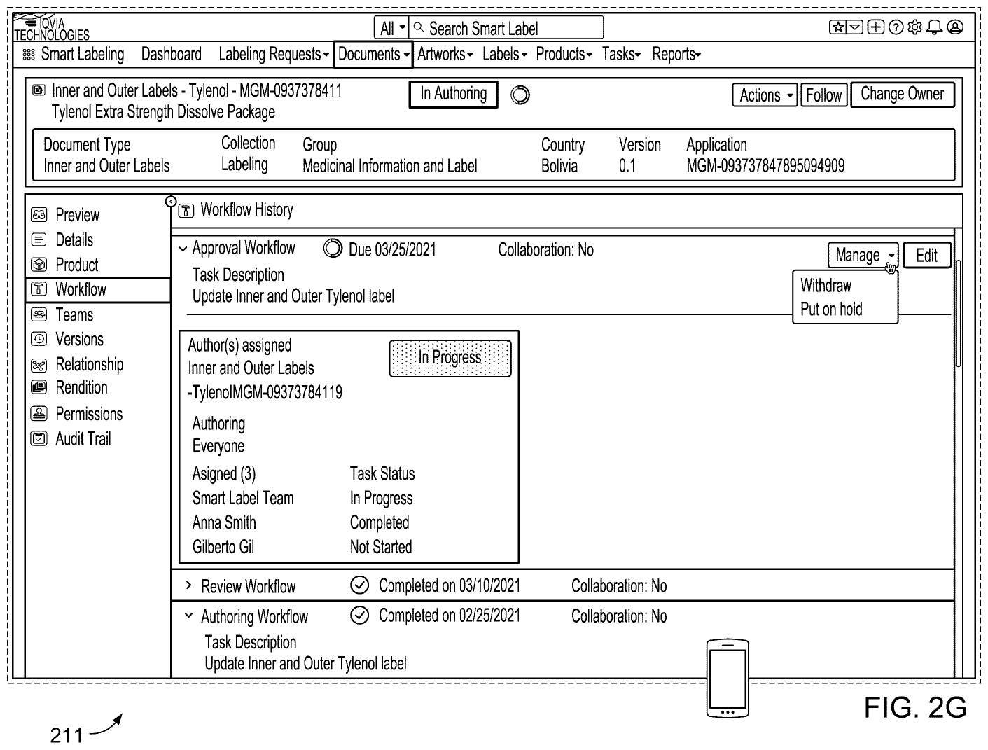

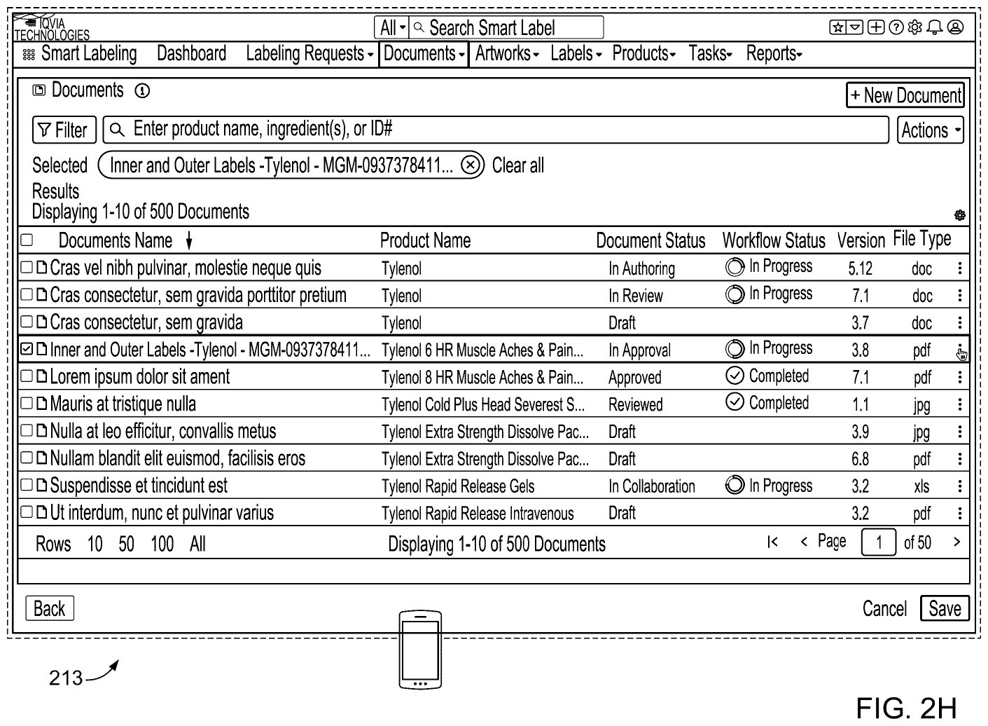

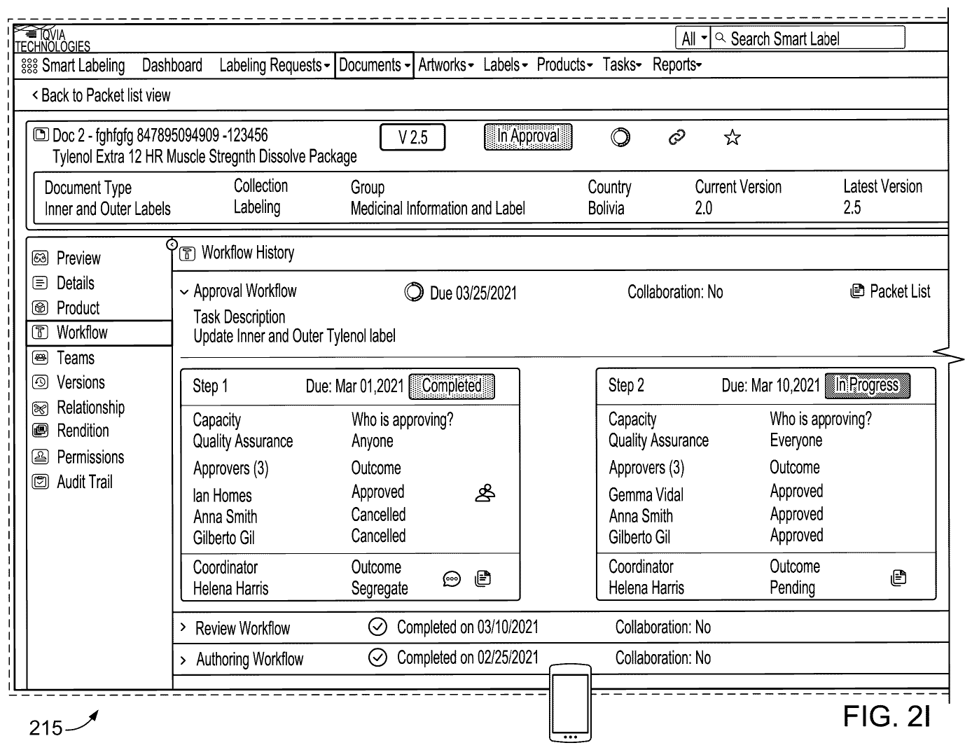

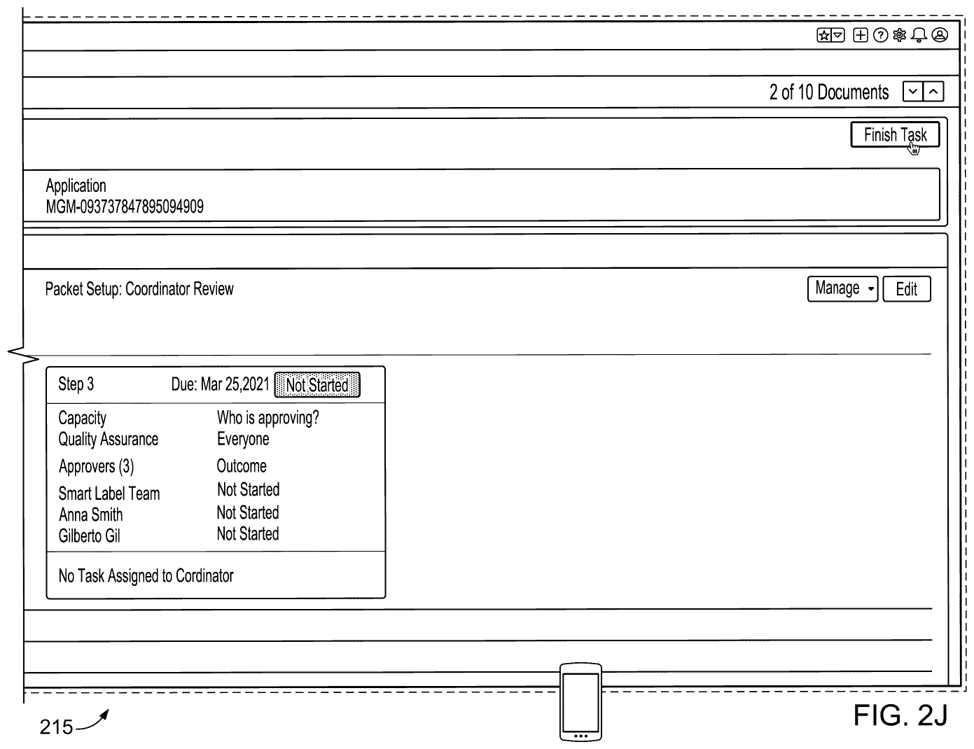

FIGS. 2A-2C are block diagrams that illustrates interfaces for interacting with a smart label management system. Specifically, FIGS. 2A, 2B, and 2C illustrate interfaces 200, 201, and 203 that display an example representation of dashboard 122, from FIG. 1A. For example, the interface 200 can connect with the interfaces 201 and 203 in a vertical or horizontal manner. As such, the interfaces 200, 201, and 203 can be displayed on a monitor connected to the management server or displayed through an application of a client device that submitted the label request. Similarly, the interfaces 200, 201, and 203 can be displayed through an application of a different client device that did not submit the label request but has access to the management server through a corresponding application.

The interface 200 illustrates techniques for tracking document lifecycles that support hybrid states. For example, the interface 200 displays one or more of the following: a FOCUS GUI, an ACTIONS GUI, an INFO GUI, a STATE GUI, a DEADLINE GUI, and a STATE GUI. The FOCUS GUI illustrates the present display on the dashboard. Here, the GUI is focused on active documents, which includes 18 different documents. The FOCUS GUI can also track active workflows and active requests. The active workflows can correspond to workflow tasks for particular documents. The active requests can track the status of label requests provided by external users. The active documents can display the documents on the dashboard which can be edited. The dashboard can illustrate the various actions related to, for example, the label request 112, the workflow process 114, the health authority submission 116, the label content localization, and the artwork management 120.

The ACTIONS GUI can enable different actions a document coordinator or user can take for each of the documents on the dashboard. These actions can include, for example, edit workflow, put on hold, withdraw, display details, or notify participants. The INFO GUI can display different information relevant to the documents. The STATE GUI can display a current state of the document. For example, the STATE GUI can include states of in authoring, authored, in review, in progress, new request, HA approved, on time, at risk, overdue, WF approved, or denied, to name some examples. The DEADLINE GUI can display a current deadline date set by the document coordinator for an assignee.

A document coordinator or user can interact with interface 200 and scroll through different workflows, documents, and requests on one single interface. Similarly, the document coordinator or user can view all the different documents in interface 201. For example, interface 201 can illustrate different documents, their versions, product, document status, and favorite documents. Similarly, interface 201 can illustrate multiple assignees and their corresponding status related to their assigned task. For example, the document coordinator can scroll with their mouse or touch a name of the assignee to view additional information about the assignee, e.g., touching “Lucas Watson” on interface 201 reveals their email address of “lucas.watson@acme.com”.

For example, with interface 200, when a particular label reaches a counter state of “Ready for Approval’ a counter increases on the dashboard. If a document coordinator is monitoring his dashboard and observes a counter increasing, the document coordinator can click on the counter, and view the labels associated with the counters increasing that are ready to proceed and initiate a workflow.

The interface 203 can be juxtaposed to interface 201, and interface 201 juxtaposed to interface 200 on the client device display. The interface 203 can display different tasks and notifications for the document coordinator. Moreover, the document coordinator can scroll through different pages of different tasks. Specifically, the interface 203 can display tasks, status of those tasks, and deadline dates related to each of those tasks that have been assigned to various assignees for a workflow. For example, one task can include “Inner and Outer Labels-Tylenol”, a status of this task includes “In Authoring,” and a deadline date of this task includes “Due Jun. 23, 2021.” In this manner, the document coordinator can review each of their corresponding tasks on interface 203 and related documents on interfaces 200 and 201.

FIG. 2D is another block diagram that illustrates an interface 205 for interacting with a smart label management system. Specifically, the interface 205 displays a planner of the dashboard. The planner can display data relevant to different personas to access snapshots across each layer of activity over time. For example, a document coordinator can review the interface 205 of the dashboard and view a timeline for the specific task of label request creation. The document coordinator can review specific details about the task.

In some examples, the planner can operate as part of a broader or multiple plans with stage gates. In further detail, the planner can involve stages of the labeling plan that are being executed outside of the labeling in another module or system. Each stage gate is API enabled, such that the opportunity to plug and play within multi-vendor landscapes is present. An example of such a plan is the EU centralized process in which labeling tracks certain stages an in others has a deliverable, e.g., such as a series of approved documents to be submitted. Then, the submission to health authority can happen in another location, and labeling is back in tracking mode waiting for the response from the health authority.

For example, the specific details can include a status of the task to be completed. The task was assigned to a developmental (dev) user to start and end on Sep. 13, 2021. Additionally, the specific details can differentiate between the planned start and end date and the actual start and end date. Following the label creation request, another task initiates and is scheduled to end of Sep. 23, 2021. In addition, the interface 205 displays multiple tasks assigned to the dev user that last from Sep. 13, 2021 to Sep. 23, 2021.

For example, the interface 205 can be displayed in the form of a Gantt chart or a timeline. A Gantt chart can display a graph with a list of activities on the left side of the chart and a predefined time scale along the horizontal portion of the chart. A horizontal bar represents each activity on the Gantt chart. The position and length of the horizontal bar represents the start date, duration, and end date of the activity. As such, by utilizing a Gantt chart for interface 205, the document coordinator can view the various activities, when each activity begins and ends, how long each activity is scheduled to last, where activities overlap with other activities and by how much, and a start and end date of an entire project that includes the various activities shown on the chart. Moreover, the Gantt chart can be interactive, e.g., click and drag stages, start and end times, by a user interacting with the interface 205. The Gantt chart can display analytics showing planned, actual, and estimated metrics for each stage, as well as variance percentages.

A Gantt chart can display a stage of a task with different cycles. For example, for a particular task, it may have taken an assignee or set of assignees three cycles to reach an approval of a task. In this example, three cycles can include the task moving from authoring to review three times, and followed by an approval. For example, the tasks shown on FIG. 2D performed by the dev user shows one example of a 3 cycle task that lopped between September 16 to the end of September 23.

FIG. 2E is another block diagram that illustrates an interface 207 for interacting with a smart label management system. Specifically, interface 207 illustrates another version of interface 205. For example, the interface 207 illustrates different activities and predicted durations of those activities. The different activities can include, for example, impact assessment, labeling committee approval, and labeling authoring and review among others.

Moreover, the interface 207 can display different durations of time related to these activities. For example, the impact assessment assigned to JC and AS is scheduled to last until November 8 and is labeled as a request stage. In another example, the impact assessment assigned to Jose is scheduled to last until November 4, is labeled as a request task, and from November 5 to November 8 is labeled as a different task. In another example, the labeling committee approval assigned to IV and MF is scheduled to last until November 14, is labeled as a request stage, and includes a designator that the labels are approved for use. Other tasks examples are also possible.

Moreover, the interface 207 illustrates dates related to the project. These dates can include an estimated start date of Nov. 1, 2020, a planned end date of Mar. 1, 2021, an estimated end date of Mar. 1, 2021, a planned duration of 120 days, and estimated duration of 120 days. This information aids the document coordinator in managing a project and managing the individuals tasks related to the project.

FIG. 2F is another block diagram that illustrates an interface 209 for interacting with a smart label management system. Interface 209 includes a detailed version of interface 207. For example, interface 209 illustrates different stage or tasks, an identifier, a risk associated with each task, a start date, an end date, a duration, a related to, a priority, and assigned to. The document coordinator can review this information to make informed decisions and adjust the information illustrated on interface 209 by, for example, adjusting tasks, adjusting assignees associated with the tasks, adjusting deadline dates, ranking the tasks by one or more of the columns, and adjusting a task's priority date. Other examples are also possible.

FIG. 2G is another block diagram that illustrates an interface 211 for interacting with a smart label management system. Specifically, the interface 211 illustrates workflow history related to the workflow processes performed at a management server. The workflow history in interface 211 illustrates, for example, author assigned for a previous workflow, various assignees for a workflow, and tasks assigned for the previous workflow.

Additionally, the interface 211 enables a user to review a workflow in progress, e.g., which corresponds to an authoring flow. The interface 211 enables a user to withdraw or put on hold a workflow in progress. The interface 211 can provide history details for previously completed workflows that indicate a completed date of previous workflows.

FIG. 2H is another block diagram that illustrates an interface 213 for interacting with a smart label management system. Specifically, the interface 213 can illustrate different documents that are currently being processed or have been previously been processed by a management server. The interface 213 can display names of each of the documents, a product name related to the document name, a document status associated with each document, a workflow status associated with each document status, a version number of the document, and a file type of the document. The file type of the document can include, for example, a word document, a PDF document, an image, e.g., JPG or PNG, and an XLS document.

FIGS. 2I and 2J are other block diagrams that illustrate an interface 215 for interacting with a smart label management system. Specifically, the interface 215 can illustrate a current workflow a document is undergoing when that document is part of a packet, e.g., a group of packets. Using interface 215, the user can view the status of each step and who is assigned to each step of the workflow. Each step of a workflow can include various tasks, those tasks assigned to various users. The user, e.g., a document coordinator or user who submitted the label request, can view other documents in the packet list and their corresponding status. The user can take action on the outcome for each tasks, after the assigned members have completed their individual tasks.

For example, the interface 215 illustrates (i) an approval workflow, (ii) a review workflow, and (iii) an authoring workflow. In the approval workflow, various steps exist, which can be ordered and ranked according to various criteria. The ordering and ranking can be based on at least one of, for example, a due date for each step, a completion of each step, and other criteria. Each step can display various approvers and their corresponding outcomes for performing each task. Each step can also display the document coordinator for each task and the corresponding outcome. Other examples are also possible.

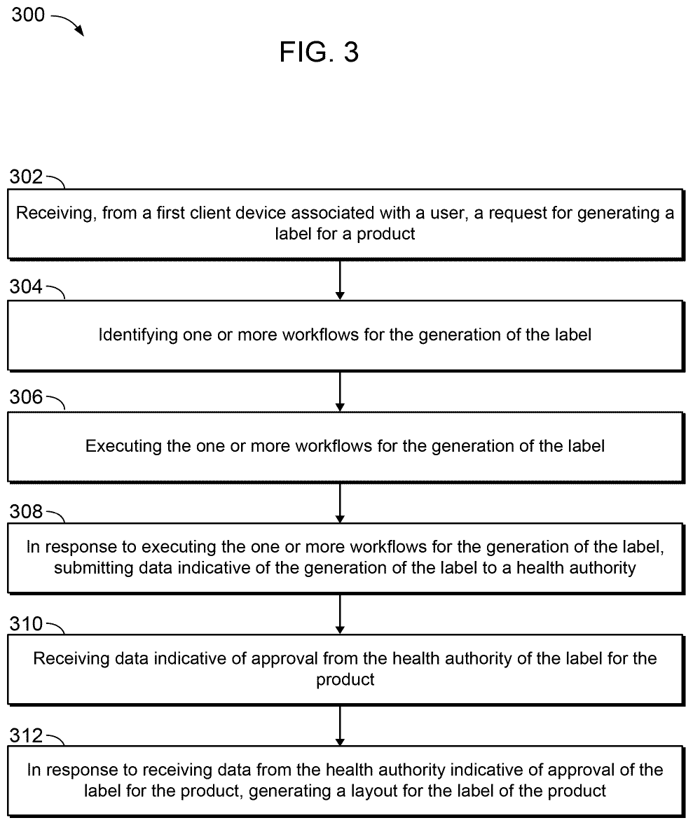

FIG. 3 is a flow diagram that illustrates an example of a process 300 for executing a label request with a smart label management system. The management server 110 can perform the process 300, for example.

The management server can receive a request for generating a label for a product (302). The management server can extract data from the request representing the label for the product. In response to extracting data from the request, the management server can submit the extracted data from the request to impact assessors to analyze socioeconomic significance of distribution of the product associated with the label. The management server can provide data indicative of a first status to a dashboard display indicating that the request has been submitted to the impact assessors. The management server can receive data indicating approval from the impact assessors for distribution of the product associated with the label. In response to receiving the data indicating of approval from the impact assessors, the management server can provide data indicative of a second status to the dashboard display indicating that the impact assessors have approved the request.

The management server can identify one or more workflows for the generation of the label (304). In further detail, the management server can assign a document coordinator to the one or more workflows for processing the generation of the label. The management server can receive, from the document coordinator, data indicative of one or more tasks for the one or more workflows, data indicative of one or more assignees for performing each of the one or more tasks, and data indicative of a length of time each of the one or more assignees has been designated for performing each of the one or more tasks. In response, the management system can determine a complexity likelihood of the one or more workflows using a trained machine-learning model.

The management server can receive as input: the data indicative of the one or more tasks for the one or more workflows, the data indicative of the one or more assignees, and the data indicative of the length of time each of the one or more assignees has been designated for performing each of the one or more tasks. In response to receiving the input, the trained machine-learning model can output, for each of the one or more tasks of the one or more workflows or for each of the one or more workflows, a complexity likelihood indicating whether a complexity of the one or more workflows is high or low. Each of the complexities may be compared to a threshold value to designate whether the complexities are a high complexity or a low complexity, e.g., by determining whether the likelihood satisfies a threshold value. If the likelihood satisfies a threshold value, e.g., meets or exceeds, then the management server can deem the complexity as a high complexity. Alternatively, the management server can deem the complexity as a low complexity.

In some implementations, the management server can segregate the workflows according to the complexity likelihood satisfying or not satisfying the threshold value. In further detail, in response to determining the complexity likelihood for each of the one or more workflows does not satisfy the threshold value, the management server can designate the one or more workflows to be performed in a multi-workflow process. Generally, the management server can designate a workflow to performed in a single-workflow process if the corresponding complexity likelihood is determined to be of low complexity. Similarly, a workflow can be performed in a multi-workflow process if the corresponding complexity likelihood is determined to be of high complexity.

The management server can execute the one or more workflows for the generation of the label (306). In some implementations, the management server can execute the one or more workflows for the generation of the label using either (i) the single-workflow process of a corresponding workflow designated as low complexity or (ii) the multi-workflow of a corresponding workflow designed as a high complexity. In response to executing the one or more workflows for the generation of the label, the management server can provide data to a dashboard display. The data provided to the dashboard display can include a status for each of (i) the one or more workflows, (ii) the layout for the label of the product, (iii) an expected time duration for completing each of the one or more workflows, and (iv) a risk associated with completing each of one or more of the workflows. The management server can provide the dashboard display to a document coordinator, a user that submitted the request, or another user monitoring the generation of the label. The management server can receive user interactions with data displayed on the dashboard display from another client device. The user interactions can include, for example, requests to view status information of workflows and other information.

In some implementations, the dashboard display can display real time updates related to the execution of the one or more workflows. The real time updates can include status information for each of i) the one or more workflows, (ii) the layout for the label of the product, (iii) the expected time duration for completing each of the one or more workflows, and (iv) a risk associated with completing each of one or more of the workflows. As each of the workflows are completed for a particular label request, the management server can receive updates from each of the authors, reviewers, or others that indicate when a workflow is completed. When each of the workflows are completed for the particular label request, the management server can prepare to submit the data indicative of the generation of the label to a health authority.

In response to executing the one or more workflows for the generation of the label, the management server can submit data indicative of the generation of the label to a health authority (308). In further detail, the management server can receive an indication that the one or more workflows have been successfully completed. As mentioned, the management server can display status updates to indicate that each of the workflows have been completed. In response, the management server can designate a health authority for submitting the data indicative of the request using the extracted data from the label request. The management server can generate a data package to submit to the health authority.

The data package can include, for example, (i) results from performing the one or more tasks for the one or more workflows, (ii) data extracted from the request for the label, and (iii) data indicative of approval from impact assessors for distribution of the product associated with the label. In response, the management server can transmit the data package to the health authority via an API, email, a website upload, or another type of submission communication. In response to submitting the data package to the health authority, the management server can provide a status to the dashboard display to indicate that the request has been successfully submitted to the designated health authority. The status may display, for example, (i) an indication of the designated health authority, (ii) an estimated time of completion for receiving a response from the health authority, and (iii) the contents of the data package that were submitted to the health authority.

The management server can receive data indicative of approval from the health authority of the label for the product (310). In some implementations, the management server can receive the data indicative of the approval from the health authority of the label. The management server can provide data indicative of a status to a dashboard display to indicate that the health authority has approved the request. Moreover, the management server can store data indicative of the user's request in a blockchain network for validation. In some cases, the management server can generate data indicative of the label from the request for distribution of the label. The data indicative of the label can include, for example, (i) artwork for the label from the request, (ii) the data indicative of the label, and (iii) a layout of the label.

In some implementations, a third party can generate the artwork for the label. In further detail, the management server can transmit the data indicative of the label from the request to a manufacturer. In response, the management server can receive data indicative of the artwork from the manufacturer and store the data indicative of the artwork in a blockchain. In some cases, the data indicative of the artwork can be used for verification purposes. The management server can compare the data indicative of the artwork generated by the manufacture to artwork stored in the block network to validate that the artwork generated by the manufacturer is being properly generated. The comparison may be performed, for example, by a machine-learning model that compares the color of pixels at each location in the compared images to one other. If the management server determines that the artwork generated by the manufacturer successfully validates to the artwork in the blockchain network, the management server can transmit a notification to the manufacturer to indicate that the artwork is successfully validated. Alternatively, if the management server determines the artwork is not successfully validated to the artwork in the blockchain network, the management server can transmit one or more edits or corrections to the artwork to the manufacturer to revise the generated artwork. In some examples, watermarking can be added to the artwork via machine learning or human processes to guarantee the origin of the document and to further prevent counterfeit versions of the artwork.

In some implementations, if the management server receives data indicating the health authority did not approve the generation of the label, the health authority can provided data indicating corrections or revisions to the label. The management server can notify the user via the dashboard that the label has not been approved and what corrections or revisions needs to be made to be subsequently approved by the health authority. The user can submit edits to the label request or a document coordinator can revise the label request so that a subsequent transmission of the revised label request to the health authority can be approved.

In response to receiving data from the health authority indicative of approval of the label for the product, the management server can generate a layout for the label of the product (312). In some implementations, the management server can generate the layout for the label to include artwork for the product, information associated with the label of the product, and other information associated with the product. The management server can provide data to a distribution team that initiates the producing of the label for a product and the distribution of the label for the product.

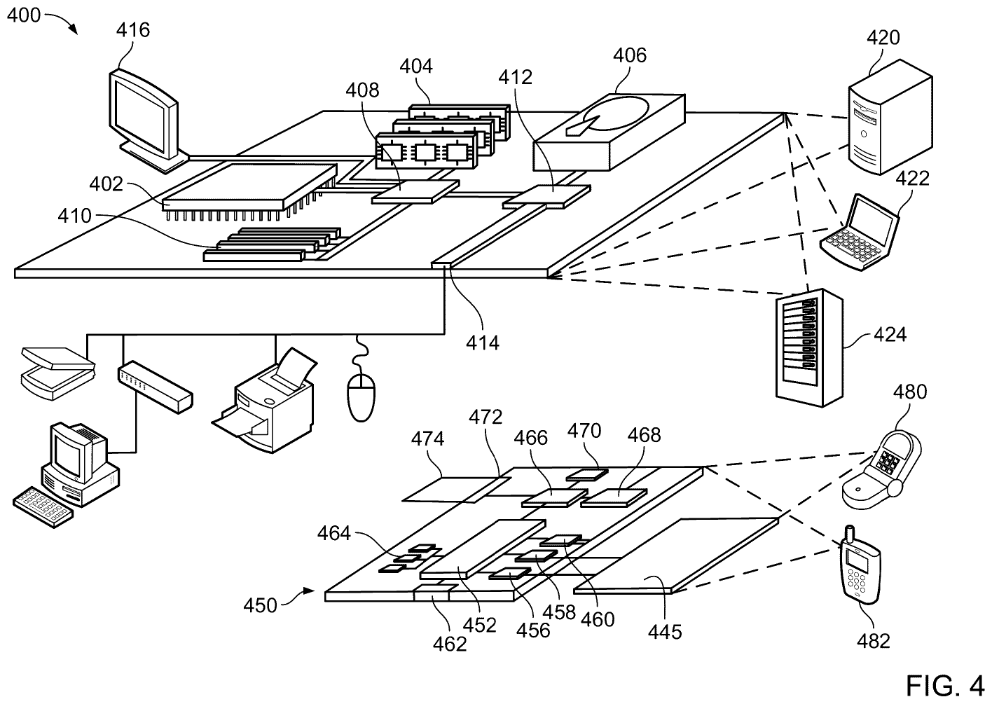

FIG. 4 is a block diagram of computing devices 400, 450 that may be used to implement the systems and methods described in this document, as either a client or as a server or multiple servers. Computing device 400 and 450 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations described and/or claimed in this document.

Computing device 400 includes a processor 402, memory 404, a storage device 406, a high-speed interface 408 connecting to memory 404 and high-speed expansion ports 410, and a low-speed interface 412 connecting to low-speed bus 414 and storage device 406. Each of the components 402, 404, 406, 408, 410, and 412, are interconnected using various busses, and may be mounted on a common motherboard or in other manners as appropriate. The processor 402 can process instructions for execution within the computing device 400, including instructions stored in the memory 404 or on the storage device 406 to display graphical information for a GUI on an external input/output device, such as display 416 coupled to high-speed interface 408. In other implementations, multiple processors and/or multiple buses may be used, as appropriate, along with multiple memories and types of memory. In addition, multiple computing devices 400 may be connected, with each device providing portions of the necessary operations, e.g., as a server bank, a group of blade servers, or a multi-processor system.

The memory 404 stores information within the computing device 400. In one implementation, the memory 404 is a computer-readable medium. In one implementation, the memory 404 is a volatile memory unit or units. In another implementation, the memory 404 is a non-volatile memory unit or units.

The storage device 406 is capable of providing mass storage for the computing device 400. In one implementation, the storage device 406 is a computer-readable medium. In various different implementations, the storage device 406 may be a floppy disk device, a hard disk device, an optical disk device, or a tape device, a flash memory or other similar solid-state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer- or machine-readable medium, such as the memory 404, the storage device 406, or memory on processor 402.

The high-speed controller 408 manages bandwidth-intensive operations for the computing device 400, while the low-speed controller 412 manages lower bandwidth-intensive operations. Such allocation of duties is exemplary only. In one implementation, the high-speed controller 408 is coupled to memory 404, display 416, e.g., through a graphics processor or accelerator, and to high-speed expansion ports 410, which may accept various expansion cards (not shown). In the implementation, low-speed controller 412 is coupled to storage device 406 and low-speed expansion port 414. The low-speed expansion port, which may include various communication ports, e.g., USB, Bluetooth, Ethernet, wireless Ethernet, may be coupled to one or more input/output devices, such as a keyboard, a pointing device, a scanner, or a networking device such as a switch or router, e.g., through a network adapter.

The computing device 400 may be implemented in a number of different forms, as shown in the figure. For example, it may be implemented as a standard server 420, or multiple times in a group of such servers. It may also be implemented as part of a rack server system 424. In addition, it may be implemented in a personal computer such as a laptop computer 422. Alternatively, components from computing device 400 may be combined with other components in a mobile device (not shown), such as device 450. Each of such devices may contain one or more of computing device 400, 450, and an entire system may be made up of multiple computing devices 400, 450 communicating with each other.

Computing device 450 includes a processor 452, memory 464, an input/output device such as a display 454, a communication interface 466, and a transceiver 468, among other components. The device 450 may also be provided with a storage device, such as a microdrive or other device, to provide additional storage. Each of the components 450, 452, 464, 454, 466, and 468, are interconnected using various buses, and several of the components may be mounted on a common motherboard or in other manners as appropriate.

The processor 452 can process instructions for execution within the computing device 450, including instructions stored in the memory 464. The processor may also include separate analog and digital processors. The processor may provide, for example, for coordination of the other components of the device 450, such as control of user interfaces, applications run by device 450, and wireless communication by device 450.

Processor 452 may communicate with a user through control interface 458 and display interface 456 coupled to a display 454. The display 454 may be, for example, a TFT LCD display or an OLED display, or other appropriate display technology. The display interface 456 may include appropriate circuitry for driving the display 454 to present graphical and other information to a user. The control interface 458 may receive commands from a user and convert them for submission to the processor 452. In addition, an external interface 462 may be provided in communication with processor 452, so as to enable near area communication of device 450 with other devices. External interface 462 may provide, for example, for wired communication, e.g., via a docking procedure, or for wireless communication, e.g., via Bluetooth or other such technologies.

The memory 464 stores information within the computing device 450. In one implementation, the memory 464 is a computer-readable medium. In one implementation, the memory 464 is a volatile memory unit or units. In another implementation, the memory 464 is a non-volatile memory unit or units. Expansion memory 474 may also be provided and connected to device 450 through expansion interface 472, which may include, for example, a SIMM card interface. Such expansion memory 474 may provide extra storage space for device 450, or may also store applications or other information for device 450. Specifically, expansion memory 474 may include instructions to carry out or supplement the processes described above, and may include secure information also. Thus, for example, expansion memory 474 may be provided as a security module for device 450, and may be programmed with instructions that permit secure use of device 450. In addition, secure applications may be provided via the SIMM cards, along with additional information, such as placing identifying information on the SIMM card in a non-hackable manner.

The memory may include for example, flash memory and/or MRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 464, expansion memory 474, or memory on processor 452.

Device 450 may communicate wirelessly through communication interface 466, which may include digital signal processing circuitry where necessary. Communication interface 466 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 468. In addition, short-range communication may occur, such as using a Bluetooth, WiFi, or other such transceiver (not shown). In addition, GPS receiver module 470 may provide additional wireless data to device 450, which may be used as appropriate by applications running on device 450.

Device 450 may also communicate audibly using audio codec 460, which may receive spoken information from a user and convert it to usable digital information. Audio codec 460 may likewise generate audible sound for a user, such as through a speaker, e.g., in a handset of device 450. Such sound may include sound from voice telephone calls, may include recorded sound, e.g., voice messages, music files, etc., and may also include sound generated by applications operating on device 450.

The computing device 450 may be implemented in a number of different forms, as shown in the figure. For example, it may be implemented as a cellular telephone 480. It may also be implemented as part of a smartphone 482, personal digital assistant, or other similar mobile device.

Various implementations of the systems and techniques described here can be realized in digital electronic circuitry, integrated circuitry, specially designed ASICs, computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs, also known as programs, software, software applications or code, include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back-end component, e.g., as a data server, or that includes a middleware component such as an application server, or that includes a front-end component such as a client computer having a graphical user interface or a Web browser through which a user can interact with an implementation of the systems and techniques described here, or any combination of such back-end, middleware, or front-end components. The components of the system can be interconnected by any form or medium of digital data communication such as, a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

In addition, certain data may be treated in one or more ways before it is stored or used, so that personally identifiable information is removed. For example, in some embodiments, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

A number of embodiments have been described. Nevertheless, it will be understood that various modifications may be made without departing from the spirit and scope of the invention. Accordingly, other embodiments are within the scope of the following claims. While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment.

Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, some processes depicted in the accompanying figures do not necessarily require the particular order shown, or sequential order, to achieve desirable results.

What is claimed is:

1. A computer-implemented method comprising:
receiving, by one or more processors and from a first client device associated with a user, a request for generating a label for a product;
identifying, by the one or more processors, one or more workflows for the generation of the label;
executing, by the one or more processors, the one or more workflows for the generation of the label;
in response to executing the one or more workflows for the generation of the label, submitting, by the one or more processors, data indicative of the generation of the label to a health authority;
receiving, by the one or more processors, data indicative of approval from the health authority of the label for the product; and
in response to receiving data from the health authority indicative of approval of the label for the product, generating, by the one or more processors, a layout for the label of the product.

2. The computer-implemented method of claim 1, wherein receiving the request for generating the label for the product further comprises:
extracting, by the one or more processors, data from the request representing the label for the product;
submitting, by the one or more processors, the extracted data from the request to impact assessors to analyze socioeconomic significance of distribution of the product associated with the label;
providing, by the one or more processors, data indicative of a first status to a dashboard display indicative that the request has been submitted to the impact assessors;
receiving, by the one or more processors, data indicating of approval from the impact assessors for distribution of the product associated with the label; and
providing, by the one or more processors, data indicative of a second status to the dashboard display indicative that the request has been approved by the impact assessors.

3. The computer-implemented method of claim 1, wherein identifying the one or more workflows for the generation of the label further comprises:
assigning, by the one or more processors, a document coordinator to the one or more workflows for processing the generation of the label;
receiving, by the one or more processors and from the document coordinator, data indicative of one or more tasks for the one or more workflows, data indicative of one or more assignees for performing each of the one or more tasks, and data indicative of a length of time each of the one or more assignees has been designated for performing each of the one or more tasks;
determining, by the one or more processors, a complexity likelihood for each of the one or more workflows using a trained machine-learning model;
comparing, by the one or more processors, the complexity likelihood for each of the one or more workflows to a threshold value;
determining, by the one or more processors, whether the complexity likelihood for each of the one or more workflows satisfies the threshold value; and
in response to determining the complexity likelihood for each of the one or more workflows satisfies the threshold value, designating, by the one or more processors, the one or more workflows to be performed in a multi-workflow process.

4. The computer-implemented method of claim 3, wherein the trained machine-learning model is configured to (i) receive as input: the data indicative of the one or more tasks for the one or more workflows, the data indicative of the one or more assignees, and the data indicative of the length of time each of the one or more assignees has been designated for performing each of the one or more tasks; and, configured to (ii) output: the complexity likelihood indicating whether a complexity of the one or more workflows is high complexity or low complexity.

5. The computer-implemented method of claim 3, wherein determining whether the complexity likelihood for each of the one or more workflows satisfies the threshold value further comprises:
determining, by the one or more processors, the one or more workflows to be of high complexity in response to determining the complexity likelihood for each of the one or more workflows satisfies the threshold value; or
determining, by the one or more processors, the one or more workflows to be of low complexity in response to determining the complexity likelihood for each of the one or more workflows does not satisfy the threshold value.

6. The computer-implemented method of claim 3, further comprising in response to determining the complexity likelihood for each of the one or more workflows does not satisfy the threshold value, designating, by the one or more processors, the one or more workflows to be performed in a single-workflow process.

7. The computer-implemented method of claim 6, wherein executing the one or more workflows for the generation of the label further comprises executing the one or more workflows for the generation of the label using either the single-workflow process or the multi-workflow process.

8. The computer-implemented method of claim 7, further comprising:
in response to executing the one or more workflows for the generation of the label further comprises:
providing, by the one or more processors and to a dashboard display, data indicative of status for each of (i) the one or more workflows, (ii) the layout for the label of the product, (iii) an expected time duration for completing each of the one or more workflows, and (iv) a risk associated with completing each of one or more of the workflows; and
receiving, by the one or more processors, user interactions with data displayed on the dashboard display from a second client device associated with the user.

9. The computer-implemented method of claim 8, wherein providing data indicative of status for each of (i) the one or more workflows, (ii) the layout for the label of the product, (iii) an expected time duration for completing each of the one or more workflows, and (iv) a risk associated with completing each of the one or more workflows, to the dashboard display further comprises:
displaying, by the dashboard display, the status for each of (i) the one or more workflows, (ii) the layout for the label of the product, (iii) an expected time duration for completing each of the one or more workflows, and (iv) a risk associated with completing each of one or more of the workflows; and
displaying, by the dashboard display, real time updates related to the execution of the one or more workflows.

10. The computer-implemented method of claim 7, wherein submitting the data indicative of the generation of the label to the health authority further comprises:
receiving, by the one or more processors, an indication that the one or more workflows have successfully completed;
designating, by the one or more processors, a health authority to submit the data indicative of the request using extracted data from the request;
generating, by the one or more processors, a data package indicative of (i) results from performing the one or more tasks for the one or more workflows, (ii) data extracted from the request for the label, and (iii) data indicative of approval from impact assessors for distribution of the product associated with the label;
submitting, by the one or more processors, the data package to the designated health authority; and
providing, by the one or more processors, data indicative of a third status to a dashboard display indicative that the request has been submitted to the designated health authority.

11. The computer-implemented method of claim 1, wherein receiving the data from the health authority indicative of approval of the label for the product further comprises:
providing, by the one or more processors, data indicative of a fourth status to a dashboard display indicative that the request has been approved by the health authority;
storing, by the one or more processors, data indicative of artwork from the request in a blockchain network for validation; and
generating, by the one or more processors, data indicative of the label from the request for distribution, wherein the data indicative of the label comprises (i) the artwork for the label from the request, (ii) the data indicative of the label, and (iii) a layout of the label.

12. The computer-implemented method of claim 11, further comprising:
transmitting, by the one or more processors, the data indicative of the label from the request to a manufacturer;
receiving, by the one or more processors, data indicative of the artwork generated by the manufacturer;
comparing, by the one or more processors, the data indicative of the artwork generated by the manufacturer to artwork stored in the blockchain network to validate that the artwork generated by the manufacturer is being properly generated; and
in response to determining that the artwork generated by the manufacturer successfully validates to the artwork in the blockchain network, transmitting, by the one or more processors, a notification to the manufacturer indicating that the artwork is successfully validated.

13. The computer-implemented method of claim 12, further comprising:
in response to determining that the artwork generated by the manufacturer does not successfully validate to the artwork in the blockchain network, transmitting, by the one or more processors, edits to the artwork being generated by the manufacturer.

14. A system comprising:
one or more computers and one or more storage devices storing instructions that are operable, when executed by the one or more computers, to cause the one or more computers to perform operations comprising:
receiving, by one or more processors and from a first client device associated with a user, a request for generating a label for a product;
identifying, by the one or more processors, one or more workflows for the generation of the label;
executing, by the one or more processors, the one or more workflows for the generation of the label;
in response to executing the one or more workflows for the generation of the label, submitting, by the one or more processors, data indicative of the generation of the label to a health authority;
receiving, by the one or more processors, data indicative of approval from the health authority of the label for the product; and
in response to receiving data from the health authority indicative of approval of the label for the product, generating, by the one or more processors, a layout for the label of the product.

15. The system of claim 14, wherein receiving the request for generating the label for the product further comprises:
extracting, by the one or more processors, data from the request representing the label for the product;
submitting, by the one or more processors, the extracted data from the request to impact assessors to analyze socioeconomic significance of distribution of the product associated with the label;
providing, by the one or more processors, data indicative of a first status to a dashboard display indicative that the request has been submitted to the impact assessors;
receiving, by the one or more processors, data indicating of approval from the impact assessors for distribution of the product associated with the label; and
providing, by the one or more processors, data indicative of a second status to the dashboard display indicative that the request has been approved by the impact assessors.

16. The system of claim 14, wherein identifying the one or more workflows for the generation of the label further comprises:
assigning, by the one or more processors, a document coordinator to the one or more workflows for processing the generation of the label;
receiving, by the one or more processors and from the document coordinator, data indicative of one or more tasks for the one or more workflows, data indicative of one or more assignees for performing each of the one or more tasks, and data indicative of a length of time each of the one or more assignees has been designated for performing each of the one or more tasks;
determining, by the one or more processors, a complexity likelihood for each of the one or more workflows using a trained machine-learning model;
comparing, by the one or more processors, the complexity likelihood for each of the one or more workflows to a threshold value;
determining, by the one or more processors, whether the complexity likelihood for each of the one or more workflows satisfies the threshold value; and
in response to determining the complexity likelihood for each of the one or more workflows satisfies the threshold value, designating, by the one or more processors, the one or more workflows to be performed in a multi-workflow process.

17. The system of claim 16, wherein the trained machine-learning model is configured to (i) receive as input: the data indicative of the one or more tasks for the one or more workflows, the data indicative of the one or more assignees, and the data indicative of the length of time each of the one or more assignees has been designated for performing each of the one or more tasks; and, configured to (ii) output: the complexity likelihood indicating whether a complexity of the one or more workflows is high complexity or low complexity.

18. The system of claim 16, wherein determining whether the complexity likelihood for each of the one or more workflows satisfies the threshold value further comprises:
determining, by the one or more processors, the one or more workflows to be of high complexity in response to determining the complexity likelihood for each of the one or more workflows satisfies the threshold value; or
determining, by the one or more processors, the one or more workflows to be of low complexity in response to determining the complexity likelihood for each of the one or more workflows does not satisfy the threshold value.

19. The system of claim 16, further comprising in response to determining the complexity likelihood for each of the one or more workflows does not satisfy the threshold value, designating, by the one or more processors, the one or more workflows to be performed in a single-workflow process.

20. A non-transitory computer-readable medium storing software comprising instructions executable by one or more computers which, upon such execution, cause the one or more computers to perform operations comprising:
receiving, by one or more processors and from a first client device associated with a user, a request for generating a label for a product;
identifying, by the one or more processors, one or more workflows for the generation of the label;
executing, by the one or more processors, the one or more workflows for the generation of the label;
in response to executing the one or more workflows for the generation of the label, submitting, by the one or more processors, data indicative of the generation of the label to a health authority;
receiving, by the one or more processors, data indicative of approval from the health authority of the label for the product; and
in response to receiving data from the health authority indicative of approval of the label for the product, generating, by the one or more processors, a layout for the label of the product.
